(19) United States
(12) Patent Application Publication
(10) Pub. No.: US 2025/0084177 A1
(43) Pub. Date: Mar. 13, 2025

(54) ANTI-CD98 ANTIBODIES AND USES THEREOF

(71) Applicant: HUAHUI HEALTH LTD., Beijing (CN)

(72) Inventors: Jianhua SUI, Beijing (CN); Xinxin TIAN, Beijing (CN)

(21) Appl. No.: 18/566,431

(22) PCT Filed: Jun. 2, 2021
(86) PCT No.: PCT/CN2021/098027

§ 371 Date: Dec. 1, 2023

(51) Int. Cl. C07K 16/28; A61K 39/00; A61P 35/00

(52) U.S. Cl. CPC C07K 16/2896; A61P 35/00; A61K 2039/505; C07K 2317/565; C07K 2317/92

(57) ABSTRACT

The present disclosure provides antibodies and antigen-binding fragments thereof that bind to mouse CD98 (mCD98) or hCD98 (hCD98). Specifically, the antibodies of the present disclosure bind to its target in a pH-dependent manner. Also provided are nucleotides encoding the antibodies or fragments thereof, compositions or combinations comprising the antibodies or fragments thereof, and methods for treating diseases such as cancers by using the antibodies or fragments thereof.

### CROSS-REFERENCE TO RELATED APPLICATIONS

This application is the U.S. national phase of International Application No. PCT/CN2021/098027 filed Jun. 2, 2021 which designated the U.S., the entire contents of which are hereby incorporated by reference.

### FIELD OF THE INVENTION

The present disclosure provides monoclonal antibodies, particularly monoclonal antibodies that specifically bind to human or mouse CD98. Also provided are nucleic acid molecules encoding the anti-CD98 antibodies, as well as expression vectors and host cells comprising the nucleic acid molecules. Pharmaceutical compositions, conjugates and multi-specific antibodies comprising the anti-CD98 antibodies are also disclosed. Also provided are methods for treating various diseases, in particular tumors, e.g. CD98-expressing tumors.

### BACKGROUND

The type II transmembrane protein CD98-which is also termed the CD98 heavy chain (CD98hc) or 4F2hc and is encoded by the SLC3A2 gene—interacts with various light chains to form diverse heterodimeric amino acid transporters (HATs). The light chains function in amino acid transport, while CD98 participates in transport activity by stabilizing the structure of light chains and facilitating their localization to the plasma membrane. CD98 has been shown to participate in tumorigenesis, tumor development, and metastasis through its promotion of amino acid transport activity, to contribute to cell survival and enhancement of integrin signaling, and to increase cell spread, migration, survival, and growth. In the past decade, CD98 has become an attractive target for developing cancer therapies because of its up-regulation in multiple types of solid and hematological malignancies and its association with poor clinical outcomes. Notably, the CD98 protein is widely expressed in normal tissues, including the brain, spleen, kidney, small intestine, testis, and hematopoietic system, etc.

In Hayes et al. (Int J Cancer 137 (2015), 710-720), an anti-human CD98 (hCD98) antibody, IGN523, was shown to exert strong antitumor activity in xenograft tumor models of leukemia, lymphoma, and lung cancer.

CN105385694B discloses a monoclonal antibody which binds to CD98 and teaches its use as a carrier to deliver anti-tumor or anti-inflammatory agents. The disclosure of CN105385694B provides only in vitro data showing that the anti-CD98 antibody is capable of binding to lung cancer cells in cell lysis, while no in vivo data is shown. The in vivo performance of the antibody, such as tumor-specific binding activity or pharmacokinetic properties is unknown.

WO2007114496A discloses multiple anti-CD98 antibodies, some of which show inhibitory effect on leucine uptake in bladder cancer cell lines and anti-tumor effect in mice bearing murine CT26 colon carcinoma line expressing hCD98/hLAT1-EGFP. One of the clones, C2IgG1, was shown to have anti-tumor effect in mice transplanted with human Burkitt lymphoma cell line Ramos.

There are other patent application publications, including AbbVie's WO2017214456A1, WO2017214458A2, WO2017214462A2, and Daiichi Sankyo Co Ltd.'s WO2015146132A1 provide conjugates comprising an anti-CD98 antibody and a drug, such as Bcl-xL inhibitors.

However, none of the above discusses the problem resulted from binding of the anti-CD98 antibody to CD98 in normal tissues. This problem does not exist in animal model bearing cancer cell lines engineered to express hCD98 or xenograft models, since the normal tissues of the animal model express murine CD98 which is not the target of the antibody. On target/off tumor binding of anti-CD98 antibodies may disrupt the function of CD98 present in normal tissue on one hand, and dilute the anti-tumor effect of the antibodies against target tumor cells on the other hand.

In fact, the widespread expression of CD98 in normal tissues poses several major challenges for clinical applications. The targeted disruption of the CD98 gene results in embryonic lethality (Tsumura et al., (2003). Biochemical and biophysical research communications 308, 847-851). Conditional knock out of CD98 impaired the proliferation and regeneration capacity of hematopoietic stem and progenitor cells (Bajaj et al., (2016). Cancer Cell 30, 792-805). Thus, on-target side effects and an “antigen sink” issue are major concerns for the development of antibody therapeutics targeting CD98.

There is an unmet need for anti-CD98 antibodies suitable as anti-tumor therapeutics. Further, anti-CD98 antibodies that have less impact on the normal physiological function of CD98 and reduce the downside of “antigen sink” phenomenon would be desirable.

### SUMMARY OF THE INVENTION

The present inventors successfully identified an anti-CD98 antibody (S1-F4) that elicits broad-spectrum Fc-dependent antitumor activity independent of disturbing CD98's physiological function in diverse xenografts and syngeneic tumors established in CD98-humanized mice. S1-F4's antitumor activity requires both innate- and adaptive-immunity components, including FcγRs, macrophages, dendritic cells, and CD8⁺ T cells. Subsequently, to overcome the “antigen-sink” problem of CD98-antibody binding in normal tissues, the present inventors solved a S1-F4/CD98 complex structure and generated a series of pH-dependent binding variants, thereby promoting overall antitumor activity by increasing tumor-specific engagement and dramatically improving the pharmacokinetics profile.

In the first aspect, the present disclosure relates to isolated antibody or an antigen-binding fragment thereof, which binds to human or mouse CD98, wherein the antibody or antigen-binding fragment thereof comprises:
a heavy chain CDR1 (HCDR1) comprising or consisting of an amino acid sequence selected from the group consisting of SEQ ID NOs:10, 20, 30, 40, 50, 60, and 70;
a heavy chain CDR2 (HCDR2) comprising or consisting of an amino acid sequence selected from the group consisting of SEQ ID NOs:11, 21, 31, 41, 51, 61, and 71;
a heavy chain CDR3 (HCDR3) comprising or consisting of an amino acid sequence selected from the group consisting of SEQ ID NOs:12, 22, 32, 42, 52, 62 and 72;
a light chain CDR1 (LCDR1) comprising or consisting of an amino acid sequence selected from the group consisting of SEQ ID NOs:15, 25, 35, 45, 55, 65 and 75;
a light chain CDR2 (LCDR2) comprising or consisting of an amino acid sequence selected from the group consisting of SEQ ID NOs:16, 26, 36, 46, 56, 66 and 76;
a light chain CDR3 (LCDR3) comprising or consisting of an amino acid sequence selected from the group consisting of SEQ ID NOs:17, 27, 37, 47, 57, 67 and 77.

In one embodiment, the antibody or fragment thereof comprises any one of following (a) to (g):
(a) HCDR1, HCDR2 and HCDR3 comprising or consisting of an amino acid sequence of SEQ ID NOs:10, 11 and 12, respectively; and LCDR1, LCDR2, LCDR3 comprising or consisting of an amino acid sequence of SEQ ID NOs:15, 16 and 17;
(b) HCDR1, HCDR2 and HCDR3 comprising or consisting of an amino acid sequence of SEQ ID NOs:20, 21 and 22, respectively; and LCDR1, LCDR2, LCDR3 comprising or consisting of an amino acid sequence of SEQ ID NOs:25, 26 and 27;
(c) HCDR1, HCDR2 and HCDR3 comprising or consisting of an amino acid sequence of SEQ ID NOs:30, 31 and 32, respectively; and LCDR1, LCDR2, LCDR3 comprising or consisting of an amino acid sequence of SEQ ID NOs:35, 36 and 37;
(d) HCDR1, HCDR2 and HCDR3 comprising or consisting of an amino acid sequence of SEQ ID NOs:40, 41 and 42, respectively; and LCDR1, LCDR2, LCDR3 comprising or consisting of an amino acid sequence of SEQ ID NOs:45, 46 and 47;
(e) HCDR1, HCDR2 and HCDR3 comprising or consisting of an amino acid sequence of SEQ ID NOs:50, 51 and 52, respectively; and LCDR1, LCDR2, LCDR3 comprising or consisting of an amino acid sequence of SEQ ID NOs:55, 56 and 57
(f) HCDR1, HCDR2 and HCDR3 comprising or consisting of an amino acid sequence of SEQ ID NOs:60, 61 and 62, respectively; and LCDR1, LCDR2, LCDR3 comprising or consisting of an amino acid sequence of SEQ ID NOs:65, 66 and 67;
(g) HCDR1, HCDR2 and HCDR3 comprising or consisting of an amino acid sequence of SEQ ID NOs:70, 71 and 72, respectively; and LCDR1, LCDR2, LCDR3 comprising or consisting of an amino acid sequence of SEQ ID NOs:75, 76 and 77;
wherein the antibody or fragment thereof comprising any one of following (a) to (f) specifically binds to hCD98, and the antibody or fragment thereof comprising (g) specifically binds to mouse CD98.

In one embodiment, the antibody or fragment thereof comprises:
a heavy chain variable region (VH) comprising an amino acid sequence having at least 80%, at least 85%, at least 90%, at least 95%, or at least 98% homology to an amino acid sequence selected from the group consisting of SEQ ID NOs:13, 23, 33, 43, 53, 63 and 73; and/or
a light chain variable region (VL) comprising an amino acid sequence having at least 80%, at least 85%, at least 90%, at least 95%, or at least 98% homology to an amino acid sequence selected from the group consisting of SEQ ID NOs:18, 28, 38, 48, 58, 68 and 78.

In a further embodiment, the antibody or fragment thereof comprises:
a VH comprising an amino acid sequence selected from the group consisting of SEQ ID NOs:13, 23, 33, 43, 53, 63 and 73, and/or
a VL comprising an amino acid sequence selected from the group consisting of SEQ ID NOs:18, 28, 38, 48, 58, 68 and 78.

In a specific embodiment, the antibody or fragment thereof comprises any one of following (a) to (g):
(a) a VH comprising or consisting of an amino acid sequence of SEQ ID NO:13, and a VL comprising or consisting of an amino acid sequence of SEQ ID NO:18;
(b) a VH comprising or consisting of an amino acid sequence of SEQ ID NO:23, and a VL comprising or consisting of an amino acid sequence of SEQ ID NO:28;
(c) a VH comprising or consisting of an amino acid sequence of SEQ ID NO:33, and a VL comprising or consisting of an amino acid sequence of SEQ ID NO:38;
(d) a VH comprising or consisting of an amino acid sequence of SEQ ID NO:43, and a VL comprising or consisting of an amino acid sequence of SEQ ID NO:48;
(e) a VH comprising or consisting of an amino acid sequence of SEQ ID NO:53, and a VL comprising or consisting of an amino acid sequence of SEQ ID NO:58;
(f) a VH comprising or consisting of an amino acid sequence of SEQ ID NO:63, and a VL comprising or consisting of an amino acid sequence of SEQ ID NO:68;
(g) a VH comprising or consisting of an amino acid sequence of SEQ ID NO:73, and a VL comprising or consisting of an amino acid sequence of SEQ ID NO:78;
wherein the antibody or fragment thereof comprising any one of following (a) to (f) specifically binds to hCD98, and the antibody or fragment thereof comprising (g) specifically binds to mouse CD98.

In one embodiment, the anti-CD98 antibody or fragment thereof has pH dependence in binding to hCD98, wherein the binding activity of the antibody or fragment thereof to hCD98 at acidic pH is higher than the binding activity at neutral pH. For example, the binding activity at acidic pH is at least 2-fold, 3-fold, 5-fold, 10-fold, 15-fold, 20-fold, 30-fold, 40-fold, 50-fold, 60-fold, 70-fold, 80-fold, 90-fold, or 100-fold higher as compared to the binding activity at neutral pH. The binding activity can be measured by any method known in the art, e.g. ELISA, FACS, or surface plasmon resonance (e.g. Biacore®). For example, when the binding activity to hCD98 is measured by ELISA assay, the EC50 of the antibody or fragment thereof at acidic pH is at least 2-fold, 3-fold, 5-fold, 10-fold, 15-fold, 20-fold, 30-fold, 40-fold, 50-fold, 60-fold, 70-fold, 80-fold, 90-fold, or 100-fold lower than the EC50 at neutral pH. The acidic pH is 5.8, 5.9, 6.0, 6.1, 6.2, 6.3, 6.4, 6.5, 6.6, 6.7 or 6.8, specifically 6.5. The neutral pH is 7.0, 7.1, 7.2, 7.3, 7.4, 7.5 or 7.6, specifically 7.4. In one embodiment, the antibody or fragment thereof has desirable binding activity at acidic pH, and has no binding activity or considerably low binding activity at neutral pH.

In one embodiment, the anti-CD98 antibody or fragment thereof shows preferential binding to hCD98 on or in tumor cells over hCD98 not on or in the tumor cells. Specifically, the binding preference is realized by the pH-dependent binding of the antibody or fragment thereof to hCD98. For example, the binding activity of the anti-CD98 antibody or fragment thereof to hCD98 at pH in a tumor microenvironment of a subject, e.g. a human, is significantly greater than the binding activity at normal physiological pH in the subject, e.g. a human. For example, when the binding activity to hCD98 is measured by ELISA assay, the EC50 of the antibody or fragment thereof at pH in tumor microenvironment is at least 2-fold, 3-fold, 5-fold, 10-fold, 15-fold, 20-fold, 30-fold, 40-fold, 50-fold, 60-fold, 70-fold, 80-fold, 90-fold, or 100-fold lower than the EC50 at normal physiological pH. In one embodiment, the pH in a tumor microenvironment can be a pH around 6.0 to 7.0, such as pH 7.0, 6.9, 6.8, 6.7, 6.6, 6.5, 6.4, 6.3, 6.2, 6.1, 6.0 or lower, e.g. pH 6.5. In another embodiment, the normal physiological pH can be the pH of normal blood or serum, e.g. pH 7.4. In one embodiment, the antibody or fragment thereof has desirable binding activity in tumor microenvironment, and has no binding activity or considerably low binding activity at normal physiological pH.

In one embodiment, the present application relates to an antibody or fragment thereof which binds to the same epitope on hCD98 with any of the above-described antibody or antigen binding fragment thereof. Specifically, the antibody or fragment thereof binds to an epitope within amino acid residues at positions 135, 376-384 and 391-399 of the amino acid sequence of hCD98 (SEQ ID NO:9). More specifically, the antibody or fragment thereof directly binds to an epitope within which amino acid residues H135, E384, E392, D397 are directly involved in binding the antibody.

In one embodiment, the anti-CD98 antibody comprises a heavy chain constant region of the subclass of IgG1, IgG2, IgG3, IgG4 or a variant thereof, and a light chain constant region of the type of kappa or lambda or a variant thereof. In a preferred embodiment, the anti-CD98 antibody comprises a heavy chain constant region of IgG1.

In one embodiment, the antigen-binding fragment is a single variable region, Fab, Fab′, F(ab′)₂, scFv, dsFv or ds-scFv.

In one embodiment, the antibody can be a multi-specific antibody, e.g., bi-specific, tri-specific antibody, a diabody or a minibody.

In a preferred embodiment, the antibody or fragment thereof comprises HCDR1, HCDR2 and HCDR3 having an amino acid sequence of SEQ ID NOs:10, 11 and 12, respectively; and LCDR1, LCDR2, LCDR3 having an amino acid sequence of SEQ ID NOs:15, 16 and 17; wherein one or more amino acids in any of the six CDRs are mutated into aspartate (D), or glutamate (E), or mutated into histidine (H), and the antibody or fragment thereof shows a pH-dependent binding to hCD98. Preferably, the amino acid mutated into D or E locates at a position in close interaction with a histidine within or near the binding epitope on hCD98, or the amino acid mutated into H locates at a position in close interaction with an aspartate or glutamate within or near the binding epitope on hCD98.

In one embodiment, the antibody or fragment thereof is used for antibody-based therapies.

In the second aspect, the present disclosure provides an isolated polynucleotide encoding the antibody or fragment thereof of the first aspect. In one embodiment, the isolated polynucleotide comprises a nucleotide sequence having at least 80% homology, at least 85% homology, at least 90% homology, at least 95% homology, at least 98% homology or 100% homology to a nucleotide sequence selected from the group consisting of SEQ ID NO:14, 19, 24, 29, 34, 39, 44, 49, 54, 59, 64, 69, 74, or 79.

In a third aspect, the present disclosure relates to an expression vector comprising the isolated polynucleotide of the second aspect.

In a fourth aspect, the present disclosure relates to a host cell comprising the isolated polynucleotide of the second aspect or the expression vector of the third aspect.

In a fifth aspect, the present disclosure relates to a composition, e.g., a pharmaceutical composition, comprising the anti-CD98 antibody or fragment thereof of the first aspect, and a pharmaceutically acceptable carrier. Preferably, the pharmaceutical composition comprises a therapeutically effective amount of the anti-CD98 antibody or fragment thereof.

In a six aspect, the present disclosure provides a method of reducing tumors, inhibiting the growth of tumor cells, treating a cancer or preventing recurrence of a cancer in a subject in need thereof, comprising administering to the subject a therapeutically effective amount of the anti-CD98 antibody or fragment thereof of the first aspect or the composition of the fifth aspect.

In a seventh aspect, the present disclosure provides a method for treating an autoimmune disease in a subject in need thereof, comprising administering to the subject a therapeutically effective amount of the anti-CD98 antibody or fragment thereof of the first aspect or the composition of the fifth aspect.

In an eighth aspect, the present disclosure provides use of the anti-CD98 antibody or fragment thereof of the first aspect in the manufacture of a medicament. In one embodiment, the medicament is used for reducing tumors, inhibiting the growth of tumor cells, treating a cancer, preventing recurrence of cancer. In one embodiment, the medicament is used for treating an autoimmune disease.

In one embodiment of the sixth or eighth aspect, the tumor or cancer expresses CD98, specifically hCD98. In one embodiment of the sixth or eight aspect, the microenvironment of the tumor or cancer has pH lower than the normal physiological pH of the subject. For example, the microenvironment of the tumor or cancer has acidic pH, such as pH 7.0, 6.9, 6.8, 6.7, 6.6, 6.5, 6.4, 6.3, 6.2, 6.1, 6.0 or lower, e.g. around pH 6.5, and the normal physiological pH of the subject is the pH of normal blood or serum, e.g. pH 7.4. More specifically, the cancer is selected from the group consisting of lymphoma, acute myelogenous leukemia, acute promyelocytic leukemia, hepatocellular carcinoma, pancreatic adenocarcinoma, pancreatic epithelioid carcinoma, breast adenocarcinoma, colorectal adenocarcinoma, skin epidermoid carcinoma, melanoma, fibrosarcoma, non-small cell lung cancer, gastric cancer, acute myeloid leukemia, glioma, tongue cancer, hypopharyngeal squamous cell carcinoma, cholangiocarcinoma, osteomalacia, osteosarcoma, renal cancer, and neuroblastoma.

In one embodiment of the seventh or eighth aspect, the autoimmune disease is caused by abnormal expansion of immune cells such as T cells or B cells. In one embodiment, the autoimmune disease is selected from multiple sclerosis, Type I diabetes or rheumatoid arthritis.

In recent study, CD98hc was identified as a robust receptor-mediated transcytosis (RMT) target which facilitates enhanced brain delivery of therapeutic antibodies (Y. Joy Yu Zuchero et al., Neuron 89, 70-82, Jan. 6, 2016). Antibodies with poor blood-brain barrier (BBB) penetration can be paired with anti-CD98 antibody as a bispecific antibody so as to improve their transport across BBB and brain accumulation.

In a ninth aspect, therefore, the present disclosure relates to a fusion protein, e.g. a bispecific antibody which comprises a first antigen-binding fragment which is the antigen-binding fragment of the first aspect that specifically binds to hCD98, and a second antigen-binding fragment that specifically binds to a second antigen different from the antigen-binding fragment of the first aspect. In one embodiment, the bispecific antibody is a therapeutic antibody or diagnostic antibody. In one embodiment, the bispecific antibody shows a higher brain accumulation as compared to a monospecific antibody that specifically binds to the second antigen after systematic administration.

In a tenth aspect, the present application relates to a combination, conjugate or composition comprising (a) the anti-hCD98 antibody of the first aspect, and (b) a second antitumor agent, in treating tumor. In one embodiment, the second antitumor agent can be an agent that regulates an immune checkpoint protein including but not limited to PD-1, PD-L1, CTLA-4, 4-1BB, 4-1BBL, CD28, CD40, CD40L, CD47, OX40, OX40L, TIM-3, TIGIT, NKG2A, B7-H3, B7-H4, VISTA, LAG3, 2B4. In one embodiment, the agent that regulates an immune checkpoint protein is an antibody that specifically binds to the immune checkpoint protein.

The present inventors found that the anti-hCD98 antibody of the present application requires macrophages and CD8+ T cells to exert its antitumor effects. Accordingly, in preferred embodiments of the tenth aspect, the second antitumor agent can be an agent enhancing the phagocytic function of macrophages, or an agent that enhancing the effect of CD8+ T cells. In one embodiment, the second antitumor agent is an agent enhancing the phagocytic function of macrophages by targeting a phagocytosis inhibitor, such as CD47. In a specific embodiment, the agent enhancing the phagocytic function of macrophages is an anti-CD47 antibody. In another embodiment, the second antitumor agent enhances the effect of CD8+ T cells, for example by blocking or reversing the negative regulation of cell-mediated immune response, e.g. by targeting an inhibitory receptor. In a specific embodiment, the second antitumor agent is an antibody that specifically binds to PD-1, CTLA-4, PD-L1, or 4-1BB. In another embodiment, the anti-CD98 antibody of the present application cannot be combined with an agent that depletes macrophages, such as an anti-CSF1R antibody.

### BRIEF DESCRIPTION OF THE DRAWINGS

FIG. 1 shows antitumor activity of HN2-G9 and IGN523 as a control in Raji tumor models. NOD SCID mice bearing Raji tumors were randomized into three groups with similar tumor volumes. Antibodies were tested at 10 mg/kg. Tumor volumes are shown as means±SEM. (n.s., not significant, *p<0.05, **p<0.01, ***p<0.001, ****p<0.0001; Two-way ANOVA). The same statistical methods and legends apply to all scatterplots with lines related to tumor volumes as described below.

FIG. 2 shows the binding specificity and avidity of IGN523, HN2-G9, and S1-F4 measured by FACS. Wild type (WT) CHO or CHO-hCD98 cells were incubated with the indicated concentrations of each antibody prior to staining with FITC-conjugated anti-human IgG secondary antibody. The binding was determined based on the fluorescence intensity for FITC.

FIGS. 3A-3F show the kinetic analysis of the binding between the indicated anti-hCD98 antibodies and hCD98 ECD, assessed via SPR.

FIG. 4 shows antitumor activity of S1-F4 in comparison to Rituximab and IGN523 as controls in Raji tumor models. NOD SCID mice bearing Raji tumors were randomized into three groups with similar tumor volumes. Antibodies were tested at 10 mg/kg.

FIG. 5 is a table listing the human cell lines for establishing xenograft tumor models.

FIG. 6 and FIG. 7 show the antitumor activity of S1-F4 in HepG2 (FIG. 6) and Ramos (FIG. 7) tumor models. CB-17 SCID mice bearing HepG2 tumors or NOD SCID mice bearing Ramos tumors were randomized into 4-5 groups with similar tumor volumes and treated with the indicated dosages of S1-F4.

FIGS. 8A-8D show antitumor activity of S1-F4 in HL60, HCT-8, BxPC-3, and PANC-1 tumor models. CB-17 SCID mice bearing HL60, BxPC-3, or HCT-8 tumors, or NOD SCID mice bearing PANC-1 tumors were randomized into two groups with similar tumor volumes: vehicle or S1-F4 (15 mg/kg). The time points for antibody treatment are marked by arrows.

FIG. 9 shows antitumor activity of S1-F4 in MDA-MB-231-LN tumor models. CB-17 SCID mice bearing MDA-MB-231-LN tumors were randomized into two groups with similar tumor volumes: vehicle or S1-F4 (15 mg/kg). Schematic diagram illustrates the timeline of tumor inoculation and S1-F4 treatment schedules. Bioluminescence imaging was conducted at 8, 18, 29, and 41 days after tumor inoculation.

FIGS. 10A-10E show antitumor activity of S1-F4 in HCT 116, A549, A-431, Hep3B, and HT-29 tumor models. CB-17 SCID mice bearing HCT 116, A549, or A-431 tumors, or NOD SCID mice bearing Hep3B or HT-29 tumors were randomized into two groups with similar tumor volumes: vehicle or S1-F4 (15 mg/kg). The time points for antibody treatment are marked by arrows. Tumor volumes are shown as means±SEM. (n.s., not significant; Two-way ANOVA).

FIGS. 11A-11F illustrate the generation of CD98 humanized mice and analysis of CD98 expression. (FIG. 11A) Schematic diagrams showing the strategy for insertion of the hCD98 ECD cassette into the mouse Slc3a2 locus. The locations of sgRNA-CD98 and indicated PCR primers in the targeted and WT alleles are shown. (FIG. 11B-11D) Gel electrophoresis of PCR reaction products from genomic DNA templates for F0 mice. The primer pairs (see Table S2) used were Y1-F/Y1-R (FIG. 11B), WT1-F/WT1-R (FIG. 11C), and IF-1/Y1-R (FIG. 11D). (FIG. 11E) Kinetics analysis of the binding of an anti-mouse CD98 antibody, BC8, to the ECD of mouse CD98 (mCD98). (FIG. 11F) The binding specificity and avidity of BC8 was measured by FACS. WT HEK293T and HEK293T-mCD98 cells were incubated with the indicated concentrations of BC8 prior to staining with FITC-conjugated anti-human IgG secondary antibody. The binding was determined based on the fluorescence intensity for FITC.

FIG. 12 shows the expression of CD98 on mice kidneys analyzed by immunofluorescence staining. Kidney samples from wild type C57BL/6 mice and CD98 humanized mice were incubated with BC8 (an mCD98-specific Ab, 15 μg/ml) or IGN523 (an hCD98-specific Ab, 15 μg/ml) prior to staining with FITC-conjugated anti-human IgG secondary antibody. Original magnification×1.3.

FIG. 13 shows the expression of CD98 on mouse leukocytes analyzed by FACS. Leukocytes from C57BL/6 mice and CD98 humanized mice were incubated with BC8 or S1-F4 (10 μg/ml) prior to staining with FITC-conjugated anti-human IgG secondary antibody.

FIG. 14 shows the expression of hCD98 and mCD98 on mice cancer cells analyzed by FACS. Cells were incubated with S1-F4 or BC8 (15 μg/ml) prior to staining with FITC-conjugated anti-human IgG secondary antibody.

FIGS. 15A-15C show antitumor activity of S1-F4 in CD98 humanized mice (50 mg/kg) (A) and C57BL/6 mice bearing syngeneic tumors (15 mg/kg) (B).

FIGS. 16A-16B show ADCC effector functions induced by S1-F4 and its Fc variants. ADCC activity was measured using LDH-release assays. Data is shown as the mean±SEM. (A) NK92-MI^{hCD16} cells were used as effector cells and Raji or HepG2 cells were used as target cells. The E:T ratio was 7:1. Antibodies were tested at the indicated concentrations. (n.s., not significant, ****p<0.0001; Two-way ANOVA). (B) Raji cells were used as target cells. Left: mBMDMs were used as effector cells. Control IgG or S1-F4 were tested at the indicated concentrations. The E:T ratio was 1:1. (****p<0.0001; Two-way ANOVA). Right: hPBMCs were used as effector cells. Vehicle or S1-F4 (10 μg/ml). The E:T ratio was 10:1. (**p<0.01; Two-tailed unpaired Student's t-test).

FIGS. 17A-17B show ADCP effector functions induced by S1-F4 and its Fc variants. (A) Raji cells (target cells) were labeled with CFSE fluorescent dye prior to mixing with mouse bone marrow-derived macrophage cells (mBMDMs) stained with anti-F4/80-Alexa Fluor 633 antibody. The E:T ratio was 1:2. ADCP activity was monitored via fluorescence microscopy and the phagocytosis index was determined as the number of CFSE-positive target cells per 100 effector cells. Scale bar, 100 μm. (B) hPBMCs were used as effector cells. Raji cells (target cells) were labeled with CFSE fluorescent dye prior to mixing with Deep-Red-labeled hPBMCs. Control IgG or S1-F4 (10 μg/ml). The E:T ratio was 1.5:1. ADCP activity was monitored via fluorescence microscopy. Representative photomicrographs are shown. Scale bar, 100 μm.

FIGS. 18A-18B show CDC effector functions (A) and binding to human C1q (B) of S1-F4 and its variants. (A) Raji cells were incubated with the indicated antibodies in the presence of 10% human complement serum. CDC activity was measured using LDH-release assays. Antibodies were tested at 10 μg/ml. (B) ELISA-based assay of S1-F4, its Fc variants, Rituximab binding to human C1q.

FIG. 19 shows the effect of S1-F4 on HPG transport in HepG2 and Raji cells. HepG2 cells or Raji cells were incubated with Control IgG (15 μg/ml), S1-F4 (15 μg/ml), or BCH (10 mM) prior to incubation in medium containing HPG (70 μM). Subsequently, the HPG present in cell lysates was biotinylated and detected with streptavidin-HRP in an ELISA-based assay. HPG uptake activity is expressed as a percentage of the HPG uptake for the Control IgG treatment group. The data is shown as the mean±SEM. (n.s., not significant, **p<0.01, ***p<0.001; Two-tailed unpaired Student's t-test).

FIGS. 20A-20D show the effect of S1-F4 on the growth of Raji, Ramos, HepG2, and HCT-8 cells. Cell growth was analyzed using WST-8 Cell Counting Kit-8. Cells were incubated with Control IgG or S1-F4 for 72 hr. The cell growth percentage is expressed as a percentage of the value detected for the untreated group. Raji cells or Ramos cells were incubated with Control IgG or S1-F4 at the indicated concentrations. (****p<0.0001; Two-way ANOVA). HepG2 cells or HCT-8 cells were incubated with Control IgG or S1-F4 (15 μg/ml). The data is shown as the mean±SEM. (n.s., not significant; Two-tailed unpaired Student's t-test).

FIGS. 21A-21D show antitumor activity of S1-F4 and its Fc variants in CD98 humanized mice bearing EL4-hCD98 or MC38-hCD98 tumors (S1-F4 or S1-F4^{DANA}, 50 mg/kg); in NOD SCID mice bearing Raji tumors or CB-17 SCID mice bearing HepG2 tumors (control IgG, S1-F4, S1-F4^{KA} or S1-F4^{DANA}, 15 mg/kg).

FIGS. 22A-22D shows that S1-F4 antibody treatment induced anti-tumor immunity upon subsequent tumor challenge. C57BL/6 mice bearing EL4-hCD98 tumors which reached complete response (CR) after S1-F4 treatment were subsequently challenged with EL4-hCD98, EL4, B16F10-hCD98, or B16F10 tumor cells at 77 days after the initial tumor cell inoculation. Age-matched naive mice were included as controls. Tumor size was assessed after (re)inoculation. Tumor volumes are shown as means±SEM. The fractions of tumor-free mice are indicated.

FIGS. 23A-23C show S1-F4 pharmacokinetics and biodistributions in monkeys and mice. Serum S1-F4 concentration vs. time profile in cynomolgus monkey and mice. S1-F4 treatment timing is marked by arrows. Serum concentrations of S1-F4 were detected using ELISA and are shown as means±SEM. Single dose of 20 mg/kg S1-F4 (i.v.) in monkeys (A); single dose of 15 mg/kg S1-F4 (i.p.) in C57BL/6 or CD98 humanized mice (B).

FIGS. 24A-24B show representative images of dissected CD98 humanized mice sacrificed 53 hr after injection of S1-F4-Cy7 (15 mg/kg) or vehicle (A). The enrichment of S1-F4 in kidneys was analyzed by immunofluorescence staining (B). C57BL/6 mice and CD98 humanized mice were treated with vehicle or S1-F4 (50 mg/kg); mice kidney samples were harvested two days later and incubated with FITC-conjugated anti-human IgG secondary antibody. S1-F4 enrichment was then determined based on the fluorescence intensity for FITC. Original magnification×1.3.

FIG. 25 shows ribbon representation of S1-F4 scFv and hCD98 ECD in orthogonal views, and detailed views of the S1-F4-CD98 interface.

FIG. 26 is an enlarged view of the interface of S1-F4 scFv and hCD98 ECD showing the position of E384, D391, E392, and D397 of CD98, and HCDR2, HCDR3, and LCDR1 of S1-F4.

FIG. 27 shows binding of S1-F4, S1-F4 Y97E, and H15L54 to hCD98 ECD at pH 6.5 or pH 7.4, analyzed with ELISA.

FIG. 28 shows the binding analysis of S1-F4, H15L1, H15L35, and H15L54 to A-431 at pH 6.5 and pH 7.4 with FACS.

FIG. 29 and FIG. 30 show that H15L54 preferentially binds to CD98 in tumors. CD98 humanized mice bearing EL4-hCD98 tumors were treated with S1-F4-Cy7 or H15L54-Cy7 (15 mg/kg). Organs were imaged at 46 hr, 90 hr, and 160 hr post-injection. (A) Representative images. (B) Quantitative tissue biodistribution. Average radiant efficiency is shown as means±SEM.

FIG. 31 shows the enrichment of indicated antibodies on mouse kidney cells of CD98 humanized mice. Mice were treated with indicated antibodies at 15 mg/kg. Mice kidney samples were harvested two days later and incubated with FITC-conjugated anti-human IgG secondary antibody. S1-F4 enrichment was then determined based on the fluorescence intensity for FITC. Images were converted to IHC images by Inform software. Original magnification×20.

FIG. 32 shows the serum antibody concentration vs. time profile in CD98 humanized mice. Mice were treated with indicated antibody (15 mg/kg, i.p.) on day 0. Serum concentrations of S1-F4 or H15L54 were detected using ELISA and are shown as means±SEM.

FIGS. 33A-33D show antitumor activity of H15L54 or S1-F4 in CD98 humanized mice bearing EL4-hCD98 tumors (FIG. 33A), MC38-hCD98 tumors (FIG. 33B), B16F10-hCD98 tumors (FIG. 33C) and CB-17 SCID mice bearing A-431 tumors (FIG. 33D). S1-F4 or H15L54 (15 mg/kg) (FIG. 33A and FIG. 33C); H15L54 (20 mg/kg) (FIG. 33B); S1-F4 or H15L54 (15 mg/kg) (FIG. 33D).

FIG. 34 shows antitumor activity of Ab8332 (8332) or S1-F4 in CD98 humanized mice bearing EL4-hCD98 tumors. Arrows indicate administration. Dosage: 15 mg/kg.

FIG. 35 shows binding of S1-F4 and Ab8332 to hCD98 ECD-His₆-Avi-Biotin at pH 6.5 and pH 7.4 measured by ELISA.

FIG. 36 shows binding of different antibodies to hCD98 ECD-His6-Avi-Biotin at pH 6.5 and pH 7.4 measured by ELISA.

FIG. 37 shows distribution of S1-F4 and Ab8332 in hCD98ECD mice bearing MC38-hCD98 tumor detected by IVIS.

### DETAILED DESCRIPTION OF THE INVENTION

Unless specifically defined elsewhere in this document, all the technical and scientific terms used herein have the meaning commonly understood by one of ordinary skill in the art to which this invention belongs.

### Materials and Methods

### Experimental Model and Subject Details

Cynomolgus monkey experiments were carried out at JOINN Laboratories (Beijing) following approved IACUC protocols.

Mouse experiments were conducted following the National Guidelines for Housing and Care of Laboratory Animals in China and performed under the approved IACUC protocols at National Institute of Biological Sciences, Beijing. C57BL/6, CB-17 SCID, and NOD SCID mice were purchased from Charles River. CD11c-DTR mice were purchased from Jackson Laboratory. CD98 humanized mice were bred and maintained in the animal care facilities at the National Institute of Biological Sciences, Beijing.

CHO or CHO-derived cell lines, HEK293T or HEK293T-derived cell lines, Raji, Ramos, HepG2, Hep3B, MDA-MB-231-LN, A549, PANC-1, EL4-hCD98, and B16F10-hCD98 cells were cultured in Dulbecco's Modification of Eagle's Medium (DMEM) supplemented with 10% fetal bovine serum (FBS). Raji, Ramos, HL60, BxPC-3, HCT-8, HT-29, HCT 116, A-431, MC38-hCD98, and MCA205-hCD98 cells were cultured with RPMI 1640 medium supplemented with 10% FBS. These cells were cultured at 37° C. in a humidified incubator with a 5% CO2 atmosphere. The FreeStyle 293F cells were from Life Technologies and were cultured following the manufacturer's instructions.

### Expression and Purification of Proteins

The CD98 ECD, or FcγRs were produced as His6-Avi-tagged fusion proteins by transient transfection of FreeStyle 293F cells and were purified by affinity chromatography. For full-length IgG antibodies, including GC33 (Ishiguro et al., 2008) and IGN523 (Hayes et al., 2015), the coding sequences of the VH and VL were subcloned into human IgG1 H chain (HC) expression vector and L chain (LC) expression vector, respectively. 293F cells were co-transfected with the two IgG expression plasmids (HC+LC plasmids) at a 1:1 ratio. After 3-6 days of transfection, the cell culture supernatants were collected for purification of IgG1 via Protein A beads affinity chromatography.

### Antibody Library Panning and Screening of Anti-CD98 Antibodies

For generation of anti-hCD98 antibodies (e.g., HN2-G9) or anti-mouse CD98 antibodies (e.g., BC8), ECD of human or murine CD98 were fused with His6-Avi tag and biotinylated by BirA ligase. These two proteins were used as antigens in the panning experiments with a human non-immune antibody library (Li et al., 2017). Phage-scFvs were screened after two rounds of selection for specific binding with CD98 ECD. A total of about 400 single clones were randomly picked and screened for binding to human or murine CD98 by ELISA. Clones selected out were produced as purified phage-scFv particles or converted into the full-length human IgG1 format for further characterizations.

### VH Chain Shuffling Sub-Library Construction and Selection

The VL gene of HN2-G9 was cloned into a phagemid vector containing a repertoire of non-immune VH genes (˜1×10¹⁰) derived from 93 healthy donors. The constructed chain shuffling library had a size of ˜1×10⁸. The library selection was done similarly as described above for antibody library selection with captured CD98 ECD-His6Avi for 3 rounds, except that HN2-G9 IgG1 was introduced as the competitor in the 3rd round of panning. A total of about 400 single clones were randomly picked and screened for binding to hCD98 by ELISA. Clones selected out were produced as purified phage-scFv particles or converted into the full-length human IgG1 format for further characterizations.

Screening of pH-Dependent Anti-hCD98 Antibodies from a Phage Display Human Non-Immune Fab Library

To screen pH-dependent antibodies, we incubated the constructed phage-Fab library with hCD98 ECD-His6-Avi-Biotin in a pH 6.5 solution, and washed with pH 6.5 washing buffer, followed by elution with pH 7.4 buffer. Then single clones were randomly picked and screened for binding to hCD98 at pH 6.5 and pH 7.4 with ELISA. Clones that bound CD98 significantly better at pH 6.5 than at pH 7.4 were converted into the full-length human IgG1 format for further characterizations.

### ELISA-Based Binding Assays

For the ELISA-based analysis of antibody binding with antigen, biotinylated protein antigens were captured with streptavidin (Sigma-Aldrich) coated 96-well plates (Nunc, MaxiSorp™). Then, serially diluted antibodies were added, and detected by adding an HRP-labeled goat polyclonal anti-Human IgG Fc (Thermo Fisher Scientific).

For the ELISA-based analysis of antibody binding with human C1q, serially diluted antibodies were coated in 96-well plates (Nunc, MaxiSorp™). Then, 3% complement sera from human (Sigma-Aldrich) was added, and detected by adding an HRP-labeled sheep polyclonal anti-Human C1q (Abcam).

### Binding Kinetic Analysis by Surface Plasmon Resonance (SPR)

Kinetic analysis of the bindings of anti-CD98 antibodies to the ECD of CD98, and the ECD of FcγRs were performed on a Biacore T200 instrument (Biacore, GE Healthcare). Anti-hFc Ab or protein A/G (Thermo Fisher Scientific) was covalently attached to the surface of a CM5 sensor chip using an amine coupling kit (GE Healthcare). Antibodies at optimal concentrations were captured on the chip and the analytes (CD98 or FcγRs) were then injected at 2-fold serial diluted concentrations. Binding kinetics were evaluated using a 1:1 Langmuir binding model. The ka, kd, and KD values were calculated using Biacore T200 evaluation software.

### Flow Cytometry Analysis of Cell Lines

For examining CD98 expression in tumor cell lines: tumor cells were stained with anti-CD98 antibodies and subsequently stained with a goat polyclonal anti-Human IgG Fc FITC (Thermo Fisher Scientific). The CD98 expression on tumor cell lines were detected by FITC.

For examining antibodies binding with CD98 variants: hCD98-GL expression plasmids containing different alanine mutations were constructed. Then CHO cells were transfected with these plasmids. Two days later, transfected CHO cells were stained with IGN523 (Hayes et al., 2015), S1-F4, or anti-GL antibody (GC33) (Ishiguro et al., (2008) Cancer Res 68, 9832-9838) and subsequently stained with a goat polyclonal anti-Human IgG Fc FITC (Thermo Fisher Scientific).

### Tissue Processing and Flow Cytometry Analyses of Immune Cells

Xenograft model mice were treated with antibodies when their tumor volume was over 500 mm3. Three days after treatment, tumors from mice were harvested and single-cell suspensions were used for FACS analyses. Briefly, tumors were dissociated and treated with Red Blood Cell Lysis Buffer. Cell suspensions were passed through a 40 μm cell strainer to obtain single-cell suspensions. Then, cells were stained with various antibodies (Key Resource Table).

### Generation of Human or Murine CD98 Expressing Cell Line

An expression plasmid was first constructed by inserting the human or murine CD98 coding DNA. The expression plasmid was then transfected into HEK293T, CHO, EL4, MC38, MCA205, or B16F10 cells. To generate cell lines stably expressing hCD98, hCD98 expressing cells were sorted with FACS after transfection and cultured in medium containing G418.

### Cell Proliferation Assay

Cell growth was analyzed using WST-8 Cell Counting Kit-8 (Dojindo Molecular Technologies). Cells (10,000-25,000 cells/well) suspended in RPMI 1640 medium containing 1% FBS were seeded in 96-well plates and incubated for 72 hr. Then CCK-8 solution (10 μl) was added to each well and the cultures were incubated at 37° C. for 1-4 hr. Absorbance at 450 nm was measured using a microplate reader. The cell growth percentage was expressed as a percentage of total untreated cells.

### Amino Acid Uptake Assay

Cells were incubated with 15 μg/ml antibody for 24 hr in growth medium. Then they were equilibrated for 30 min at 37° C. with Met-free DMEM. HPG (Thermo Fisher Scientific) was added to the cells at a final concentration of 70 μM. BCH (2-amino-2-norbornane-carboxylic acid) (Sigma-Aldrich) was added as positive control at the same time as HPG at a final concentration of 10 μM. After 2-3 hr, cells were washed with PBS and lysed with lysis buffer (1% SDS in 50 mM Tris-HCL, pH 8.0) with complete protease inhibitors (Roche). HPG in cell lysis was biotinylated according to manufacturer instructions (Thermo Fisher Scientific). Then cell lysis was transferred into 96 well plate (Nunc, MaxiSorp™) and incubated at 4° C. overnight. The amount of HGP in cell lysates was then detected by streptavidin-HRP (Thermo Fisher Scientific) in an ELISA-based assay.

### ADCC, ADCP, and CDC Assays

For the ADCC assay, target cells (10,000-20,000 cells/well) were seeded into the wells of U-bottomed 96-well cell culture plates and incubated briefly with various concentrations of different antibodies. The effector cells were then added (E:T=4:1−10:1) into the wells containing the target cells and antibodies, and incubated for 4-8 hr at 37° C. in RPMI 1640 medium supplemented with 5% FBS. ADCC activity was determined by lactate dehydrogenase (LDH) release following the instructions of a CytoTox 96® Non-Radioactive Cytotoxicity Assay kit (Promega). Percentage cytotoxicity was calculated following the manufacturer's instructions.

For ADCP assays, mouse bone marrow-derived macrophage cells (mBMDMs) and human peripheral blood mononuclear cells (hPBMCs) were used as effector cells. To prepare mBMDMs, mouse bone marrow cells were collected from the tibia and femurs of C57BL/6 mice, and induced by GM-CSF in L929 supernatants for three days. The Raji cells was labeled with CFSE (Thermo Fisher Scientific) and used as target cells. The mBMDMs were labeled with anti-mouse F4/80-Alex Fluor647 (Thermo Fisher Scientific) prior to incubation with target cells. The CFSE-labeled target cells were incubated with different antibodies at room temperature for 15 min and then added to the labeled mBMDMs in an ET ratio of 1:2 for 2 hr at 37° C. in DMEM medium supplemented with 10% heat-inactivated FBS. Phagocytosis of CFSE-labeled target cells by anti-mouse F4/80 Ab-labeled macrophages was recorded using a Nikon AIR Confocal Microscope. hPBMCs differentiation was induced by 20 ng/mL macrophage colony-stimulating factor (M-CSF) (PeproTech) for nine days before use as effector macrophages. ADCP assays with hPBMCs were similar with those described above, except that the hPBMCs were labeled with Deep Red Dye (Thermo Fisher Scientific).

For the CDC assay, target cells were seeded in a 96-well U-bottomed plate at 400,000 cells/well, incubated with various antibodies in the presence of 5% human sera (Sigma-Aldrich). After 2 hr of incubation, the supernatants in each well were analyzed for LDH release using a CytoTox 96® Non-Radioactive Cytotoxicity Assay kit (Promega).

### Pharmacokinetics and Safety Assessment of S1-F4 in Monkey

Healthy cynomolgus monkeys (3-4 years old) weighing approximately 3 kg were intravenously (i.v.) injected with S1-F4 on Day 0 and Day 15. Blood samples were collected at different time points and serum concentrations of antibody were measured with a hCD98-binding ELISA. Then antibody serum concentration was calculated and plotted in Graphpad Prism 6. The evaluation of the PK data was conducted with WinNonlin software. Body weight, body temperature, blood biochemistry (alanine transaminase (ALT), aspartate transaminase (AST), and creatinine), red blood cell content (RBC), and white blood cell content (WBC) were assessed at pre-determined timepoints at JOINN Laboratories (Beijing).

### Pharmacokinetics Analysis in Mice

6-8-week old C57BL/6 or CD98 humanized mice were used in this experiment. Blood was collected at different timepoints after a single intraperitoneal (i.p.) injection of analyte antibodies. Antibody concentrations in serum were measured with a human IgG ELISA quantitation kit (Bethyl Laboratories). Then antibody serum concentration was calculated and plotted in Graphpad Prism 6. The evaluation of the PK data was conducted with WinNonlin software.

Structural Characterization of the S1-F4 scFv and hCD98 ECD Complex

For X-ray crystallography analysis, S1-F4-scFv-His6 and hCD98 ECD recombinant protein were used. Amino acid sequence of hCD98 ECD corresponds to residues Glu111-Ala529 of hCD98. S1-F4-scFv-His6 was expressed in FreeStyle 293F cells and hCD98 ECD was expressed in E. coli. S1-F4-scFv-His6 was purified by Immobilized Metal Ion Affinity Chromatography (IMAC) using Ni-NTA agarose beads (QIAGEN), and hCD98 ECD was purified by same beads followed by HiTrap Q HP anion exchange chromatography column (GE Healthcare). Then S1-F4-scFv-His6 and hCD98 ECD were mixed to form a complex and purified by Size Exclusion Chromatography with Superdex S200 10/300 GL column (GE Healthcare). The purified S1-F4-scFv-His6/hCD98 ECD complex was then concentrated and crystallized at 20° C. using the hanging-drop vapor-diffusion method by mixing 1 μL of protein (10 mg/mL in 10 mM Tris-HCl pH 8.0 and 150 mM NaCl) and 1 μL of reservoir solution containing 0.1 M sodium citrate tribasic dihydrate pH 5.0, 9% PEG20000, 5% PEG400, 9% glycerol.

Plate-shaped crystals appeared after 7 days. The X-ray diffraction data were collected at the Shanghai Synchrotron Radiation Facility beamline BL19U1 and processed by XDS. The structure was determined at 2.8 Å resolution by molecular replacement in Phaser using the structure of hCD98 ECD (PDB 2DH2) as starting model. The initial model from molecular replacement was further refined in Phenix and manually rebuilt with Coot. The final model includes 235 residues of S1-F4 scFv and residues 115-526 of the hCD98 ECD. MolProbity analysis showed that 96.55% of residues are in the favored region and 3.41% of residues are in the allowed region.

### In Vivo Tumor Assays

For mouse tumor models, 6-8-week old mice were inoculated subcutaneously with various tumor cells (in 100 μL DPBS or medium). Except for MDA-MB-231-LN cells inoculated on the breast fat pad of female mice, all other cells were inoculated on the right flank. For xenograft tumor models, based on similar mean tumor volumes, mice were randomized into groups (n=2-6/group) and received intraperitoneal injection of various antibodies. For syngeneic tumor models, mice were randomized into groups (n=3-10/group) after tumor cells inoculation (1×10⁵ cells in DPBS) and received intraperitoneal injection of various antibodies. Tumor volume was measured with an electronic caliper and calculated using the modified ellipsoid formula ½×(length×width2). When the tumor reached 2 cm in length or when weakness was observed, the mice were sacrificed. Additional information regarding the xenograft tumor models is listed in Table 1 below.

TABLE 1
Establishment of xenograft tumor models

| Cell line | Mice strain | Inoculation method |
|---|---|---|
| Raji | NOD SCID | 1 × 10⁶ cells in DPBS |
| Ramos | NOD SCID | 5 × 10⁶ cells in DPBS |
| HL60 | CB-17 SCID | 1 × 10⁷ cells in DPBS |
| HepG2 | CB-17 SCID | 5 × 10⁶ cells in DMEM (with 50% Matrigel) |
| Hep3B | NOD SCID | 5 × 10⁶ cells in DMEM |
| BxPC-3 | CB-17 SCID | 5 × 10⁶ cells in RPMI1640 |
| MDA-MB- | NOD SCID | 5 × 10⁶ cells in DPBS (with 50% Matrigel) |
| 231-LN | | |
| HCT-8 | CB-17 SCID | 5 × 10⁶ cells in RPMI1640 |
| HT-29 | NOD SCID | 5 × 10⁶ cells in DPBS |
| HCT 116 | NOD SCID | 5 × 10⁶ cells in DPBS |
| A-431 | CB-17 SCID | 5 × 10⁶ cells in RPMI1640 |
| A549 | CB-17 SCID | 3 × 10⁶ cells in DMEM |
| PANC-1 | NOD SCID | 4 × 10⁵ cells in DPBS (with 50% Matrigel) |

F or depletion of CD4+ or CD8+ T cells, tumor-bearing CD98humanized mice were injected with 15 mg/kg of anti-CD4 antibody (clone GK1.5, BioXCell) or 10 mg/kg of anti-CD8α antibody (clone 2.43, BioXCell) one day before S1-F4 treatment and once every 3-5 days. For depletion of NK cells, mice were injected with 2.5 mg/kg anti-Asialo-GM1 polyclonal antibody (Poly21460, Biolegend) one day before S1-F4 treatment and once every six days. For depletion of neutrophils, mice were injected with 20 mg/kg of anti-Ly6G antibody (clone 1A8, BioXCell) one day before S1-F4 treatment and once every three days. For depletion of macrophages, mice were injected with 25 mg/kg of anti-CSF1R antibody (clone AFS98, BioXCell) 1-2 days before S1-F4 treatment and once every three days. For depletion of dendritic cells, mice were injected with 4 μg/kg of Diphtheria Toxin (Sigma-Aldrich) one day before S1-F4 treatment and once every two days. The efficiencies of the immune cell depletion methods described above were confirmed by using tumor-naive mice.

For tumor re-challenge studies, C57BL/6 mice that displayed complete response (CR) of EL4-hCD98 tumors upon S1-F4 treatment and age-matched naive C57BL/6 mice were inoculated with 1×10⁵ tumor cells into flanks (inoculating EL4 and EL4-hCD98 on the left flank, and inoculating B16F10 and B16F10-hCD98 on the right flank). Tumors were measured as described above.

### Immunofluorescence Staining Assays

For detecting enrichment of antibodies in mice kidneys, mice were sacrificed 2-3 days after injection with various antibodies. Then mice kidneys were harvested to prepare frozen sections. Kidney sections were stained with a goat polyclonal anti-Human IgG Fc FITC (Thermo Fisher Scientific). The CD98 expression on the surface of kidney cells was detected by FITC with a Vectra Polaris instrument (PerkinElmer).

For examining CD98 expression in tumors, tumors were harvested to prepare frozen sections. Tumor sections were then stained with BC8 or IGN523 (10 μg/ml) followed with a goat polyclonal anti-Human IgG Alexa Fluor 633 (Thermo Fisher Scientific). The CD98 expression on the surface of tumor cells was detected by Alexa Fluor 633 with a Nikon A1R Confocal Microscope.

For examining CD98 expression on mice tissues, various mice tissues were harvested to prepare frozen sections. Sections were then stained with BC8 or IGN523 (10 μg/ml) and subsequently stained with a goat polyclonal anti-Human IgG Fc FITC (Thermo Fisher Scientific). The CD98 expression in mouse tissues was detected by FITC.

### Optical Imaging of Tumor-Bearing Mice

Optical Imaging of tumor-bearing mice was performed with an IVIS Spectrum instrument (PerkinElmer) and analyzed with Living Image 4.4 software (PerkinElmer). Identical illumination settings were used for acquiring all images of one experiment, and fluorescence emission was normalized, as is common in bioluminescence imaging.

For fluorescence imaging of antibody-Cy7 distribution in CD98 humanized mice, tumor-bearing CD98 humanized mice (n=2-3/group) were i.p. injected with vehicle or Cy7-labeled antibodies (S1-F4-Cy7 (Cy7/antibody=0.845) or H15L54-Cy7 (Cy7/antibody=1.076)). Mice were dissected and fluorescence images were obtained at various timepoints. The fluorescence images were acquired using an IVIS Spectrum instrument equipped with 745 nm excitation and 800 nm emission filters.

For bioluminescent imaging of MDA-MB-231-LN tumor models. MDA-MB-231-LN-tumor-bearing mice were injected (i.p.) with 150 mg/kg of D-luciferin (PerkinElmer). Tumor bioluminescence was determined 10 min after D-luciferin injection. Imaging was performed every 4-5 days until the last day on which all mice in all groups were alive.

### Statistical Analysis

GraphPad Prism 6 (GraphPad) was used for specific comparisons throughout the manuscript with p values indicated in the relevant figure legends. Two-way ANOVA or two-tailed unpaired Student's t-tests were applied. Two-way ANOVA was applied to analyzing antitumor activity. p values<0.05 were regarded as statistically significant; (n.s., not significant, *p<0.05, **p<0.01, ***p<0.001, ****p<0.0001).

### EXAMPLES

### Example 1. Generation of Anti-hCD98 Antibody S1-F4

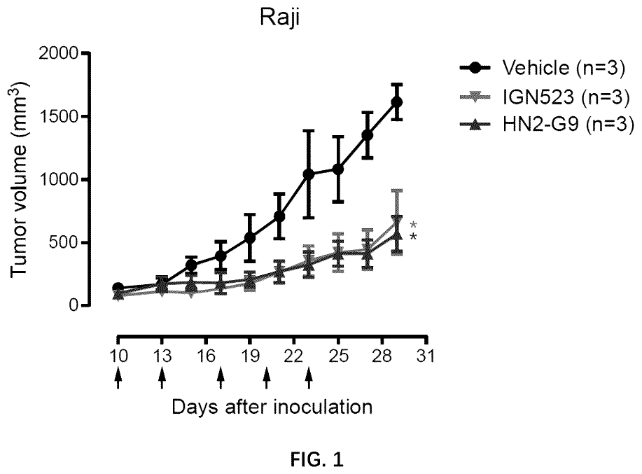

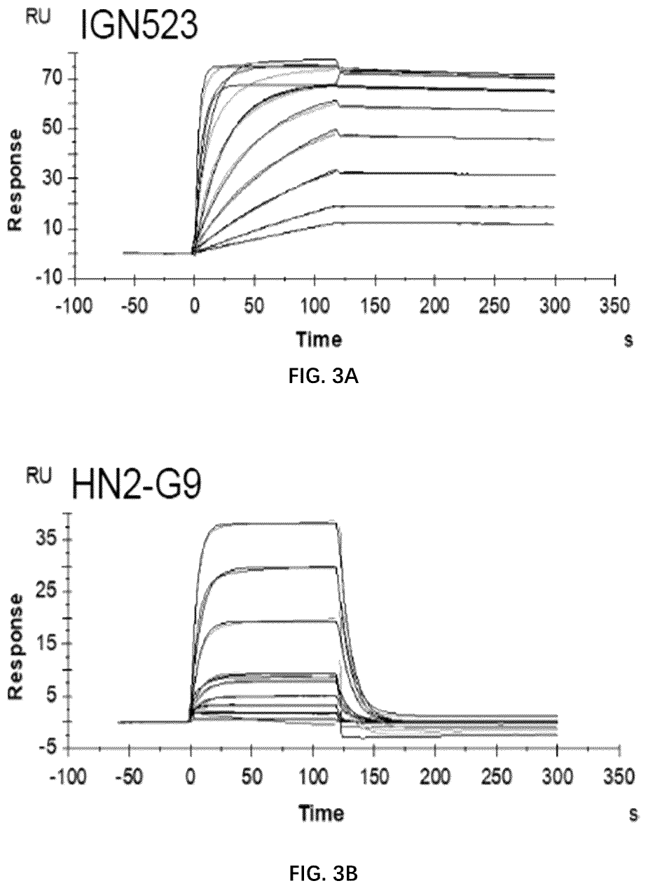

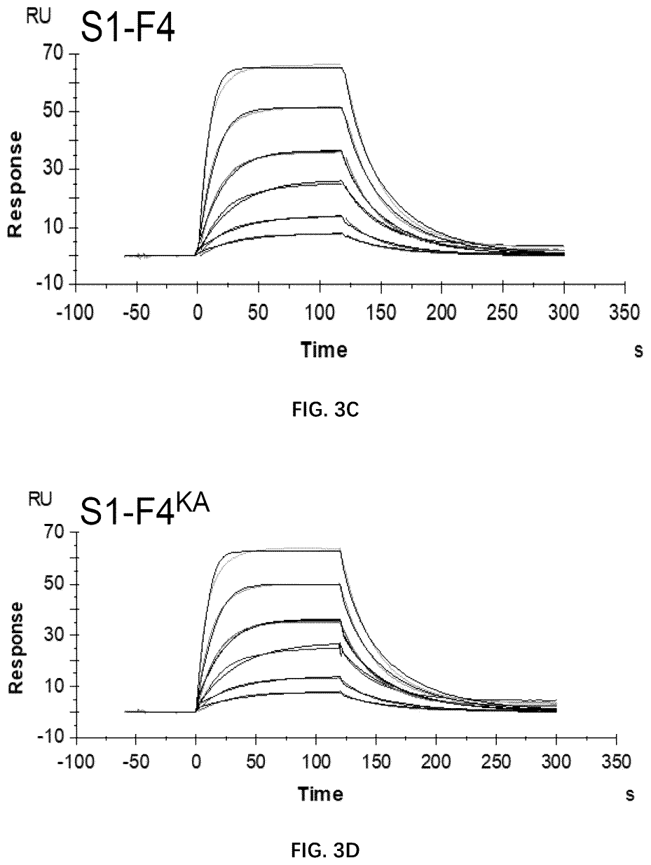

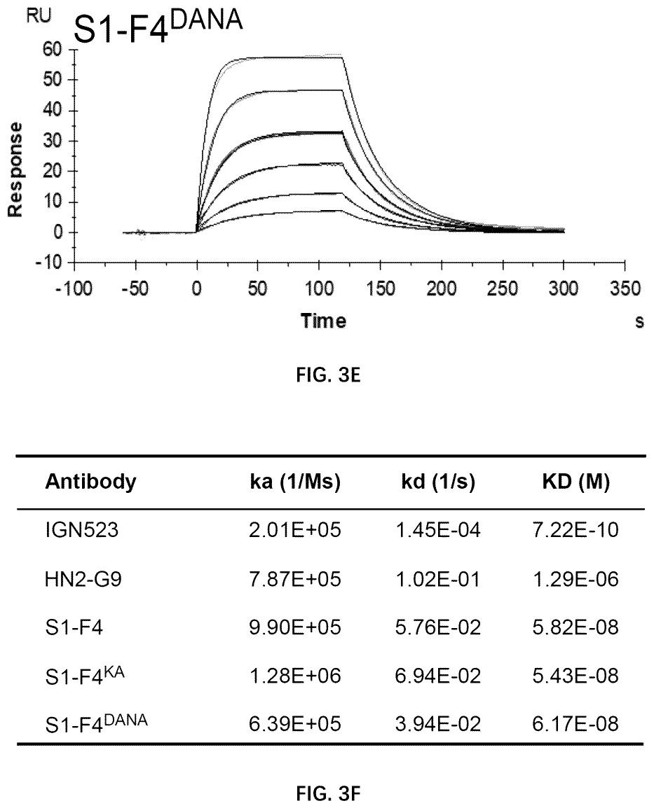

A recombinant protein containing the extracellular domain of hCD98 (hCD98 ECD) was produced for selection of anti-hCD98 antibodies from a non-immune phage display single-chain Fv (scFv) human antibody library (Li et al., (2017) Elife 6.). FACS-based analysis of antibody binding to hCD98-expressing CHO cells (CHO-hCD98) was conducted to screen the top-performing antibody. HN2-G9 was identified out of seven candidates and evaluated for its antitumor activity using a xenograft mouse tumor model (Raji, Burkitt's lymphoma). HN2-G9 elicited comparable antitumor activity as the aforementioned prior-art anti-CD98 antibody, IGN523 (Hayes et al., 2015) (FIG. 1). However, surface plasmon resonance (SPR) analysis indicated that HN2-G9 had relatively low binding affinity (1.29 μM) for CD98 as compared to IGN523 (FIGS. 3A-3F).

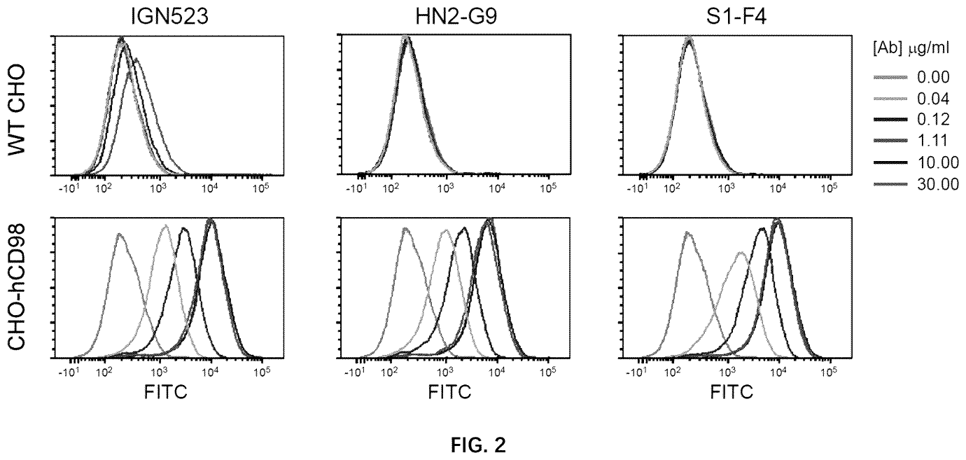

Seeking to improve the CD98 binding affinity of HN2-G9, HN2-G9 was engineered by using a variable heavy (VH) chain shuffling approach (Li et al., 2017). One antibody (S1-F4) was identified with significantly higher binding affinity in both FACS and SPR analysis (58.2 nM) compared to HN2-G9 (FIG. 2, FIGS. 3A-3F).

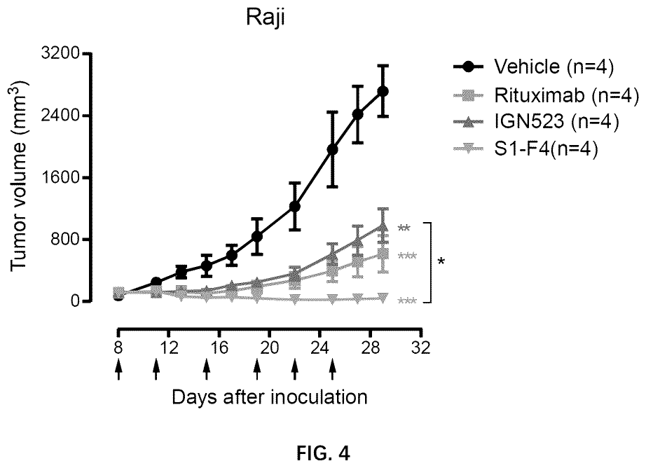

Subsequent testing of S1-F4's performance against the Raji xenograft tumors showed that it exerted significantly stronger antitumor effects than IGN523 and similar antitumor effects as Rituximab (an FDA-approved anti-CD20 antibody for treating B-cell lymphoma) (FIG. 4).

### Example 2. Broad-Spectrum Antitumor Activity of S1-F4 in Xenograft Tumor Models

As CD98 is highly expressed in many cancer types, S1-F4's antitumor effects were evaluated in various xenograft tumor models.

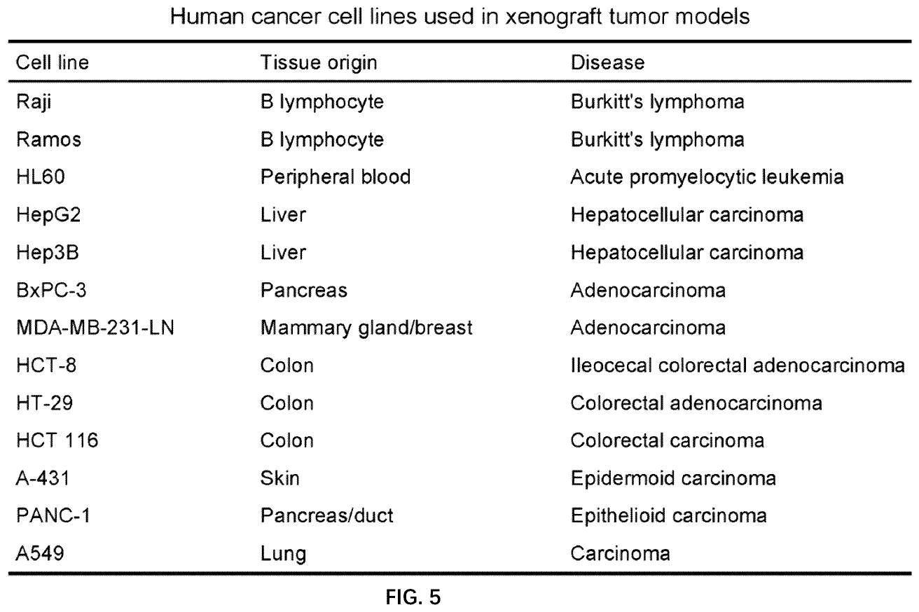

Prior to in vivo studies, CD98 expression in 13 human tumor cell lines (including Raji) from different tissue origins was examined (FIG. 5). All of the tested cell lines expressed high levels of CD98. Thus, xenograft tumor models were established using these cell lines in immune-deficient CB-17 SCID or NOD SCID mice to evaluate S1-F4's therapeutic effects.

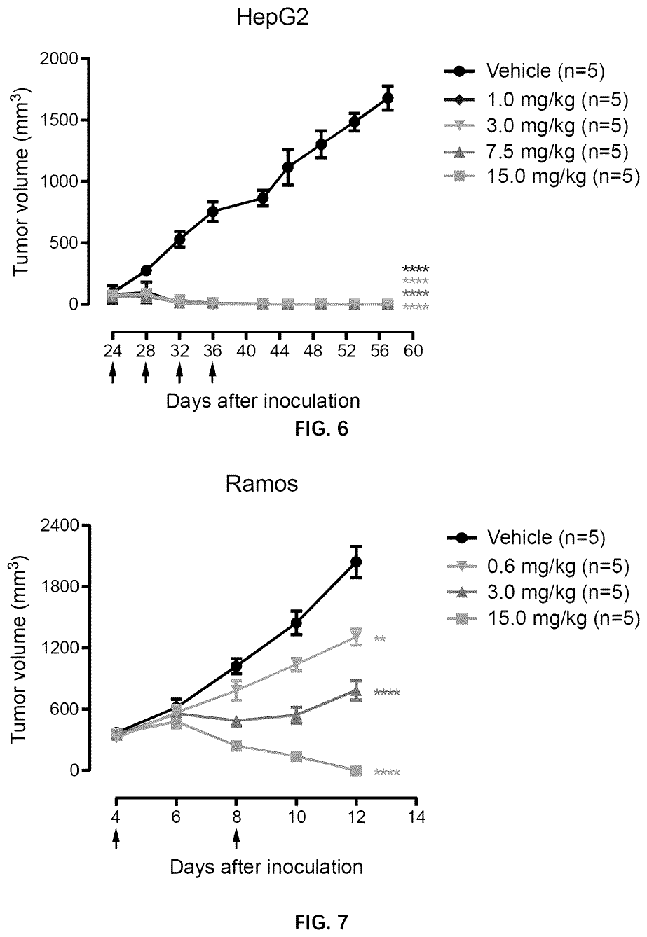

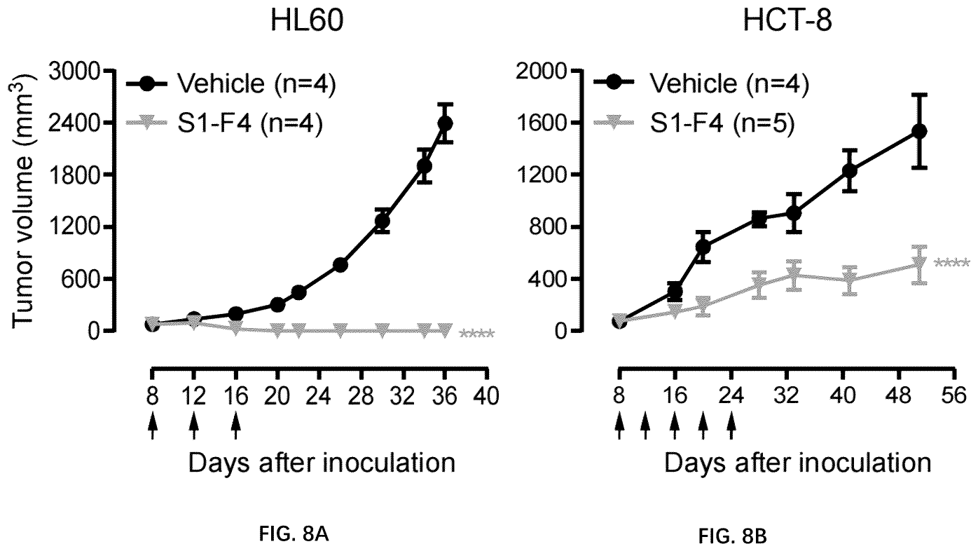

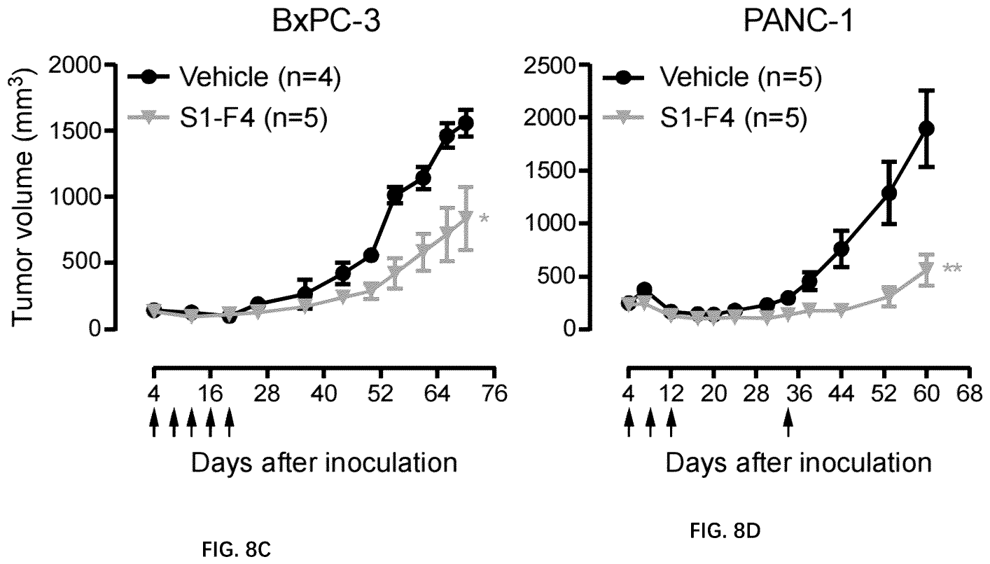

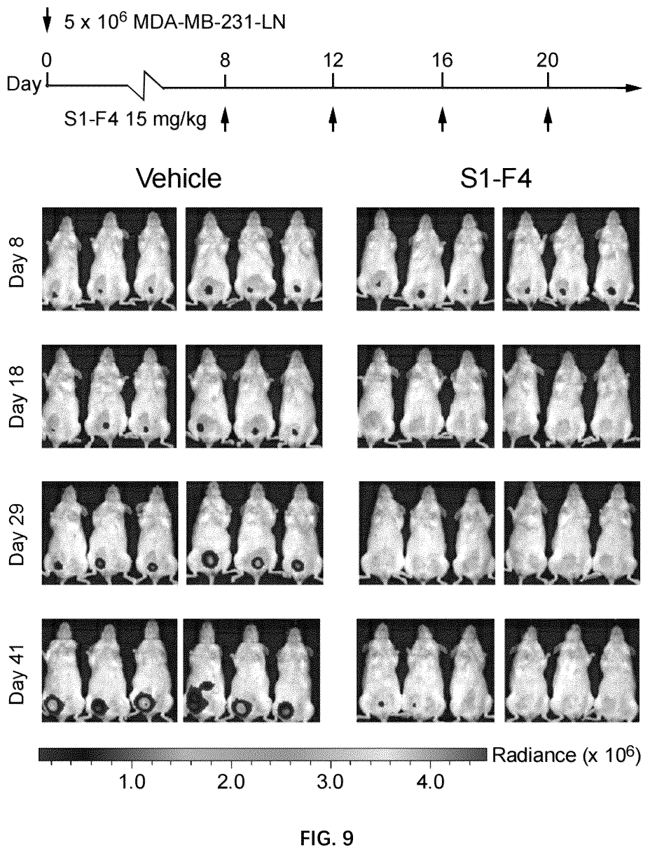

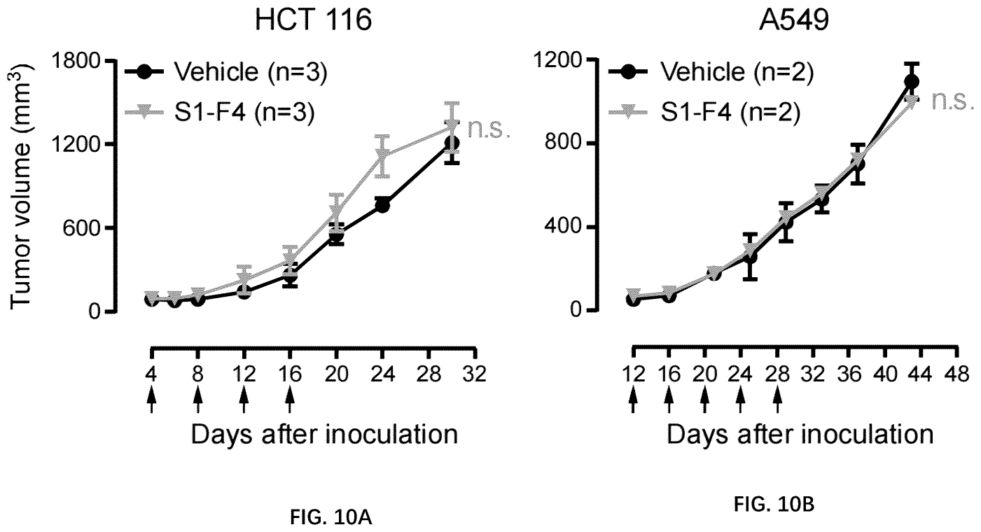

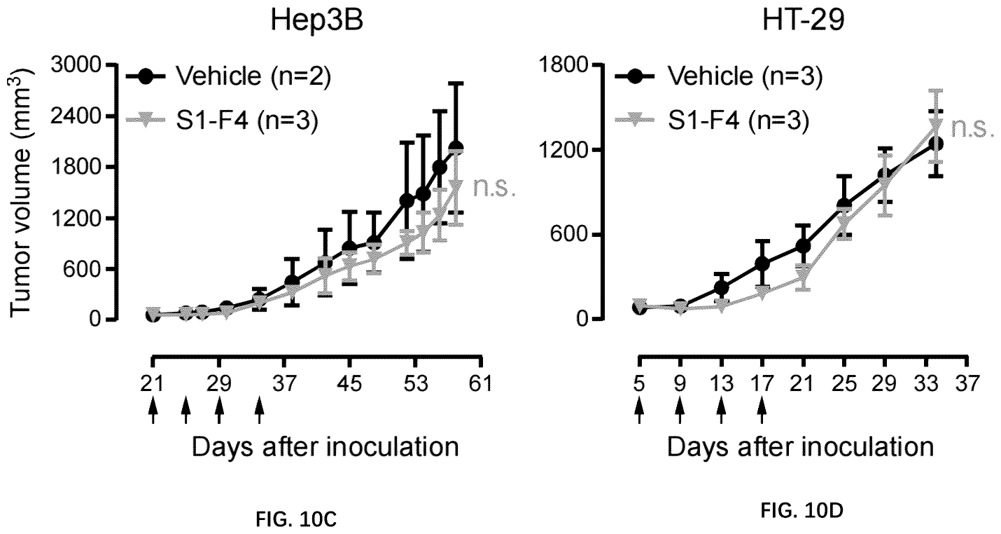

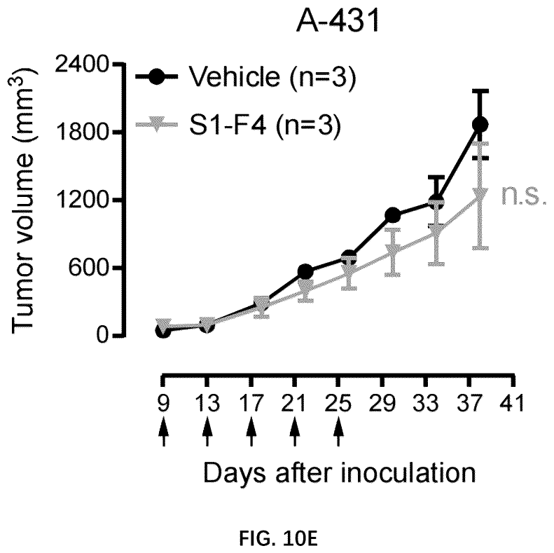

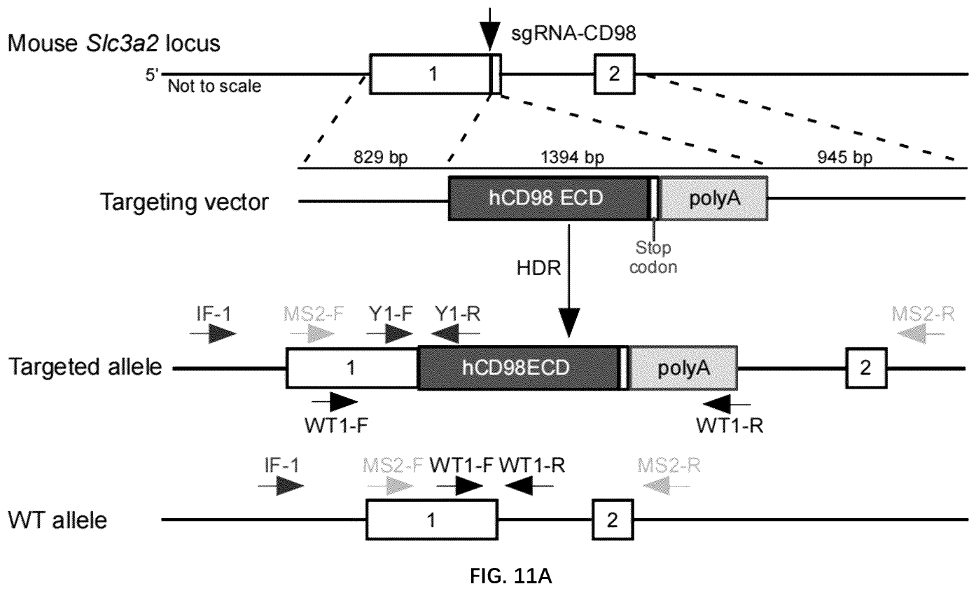

S1-F4 treatment showed broad antitumor effects in 8 out of the 13 xenograft models (FIGS. 4, 6-10). Of particular note, S1-F4 treatment at a low (1 mg/kg) dose resulted in complete tumor regression in the HepG2 (hepatocellular carcinoma) tumor model (FIG. 6).

### Example 3. Generation of Mouse Model Expressing hCD98 (CD98 Humanized Mice)

It was found that S1-F4 binds to human and monkey CD98 but not mouse CD98 (mCD98) (Table 1). Given the widespread expression of CD98 in many normal tissues of human, it is very likely that the results from the aforementioned mouse models with human xenografts could not accurately reflect S1-F4's potential effects and/or safety profile for humans.

TABLE 1
Binding affinity of S1-F4 to CD98 of different origins

| | ka (1/Ms) | kd (1/s) | KD (M) |
|---|---|---|---|
| Human | 9.90E+05 | 5.76E−02 | 5.82E−08 |
| Monkey | 4.69E+05 | 8.82E−02 | 1.88E−07 |
| Mouse | n.m. | n.m. | n.m. |

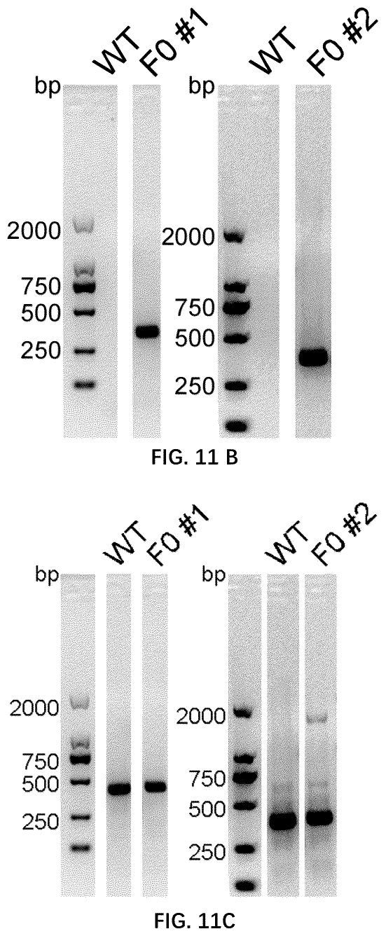

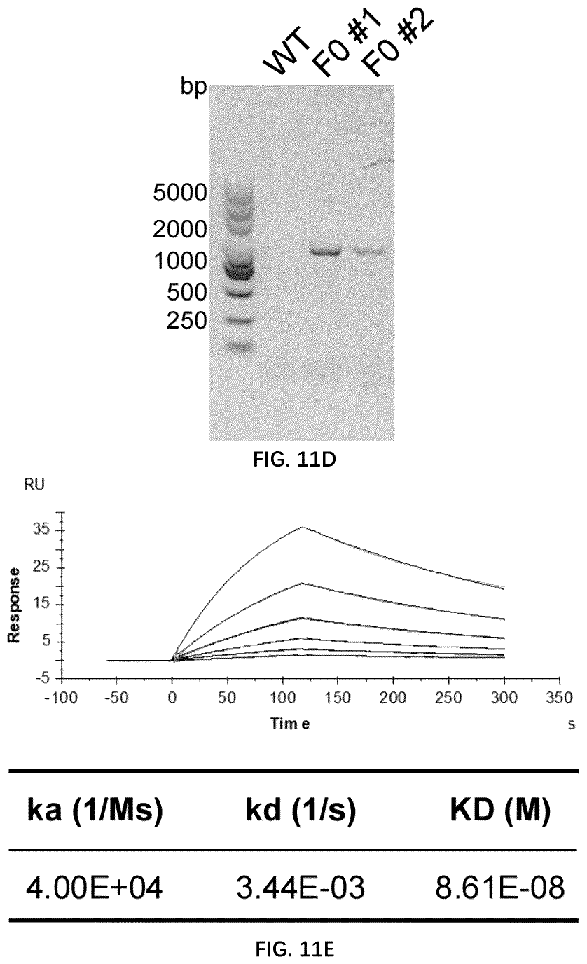

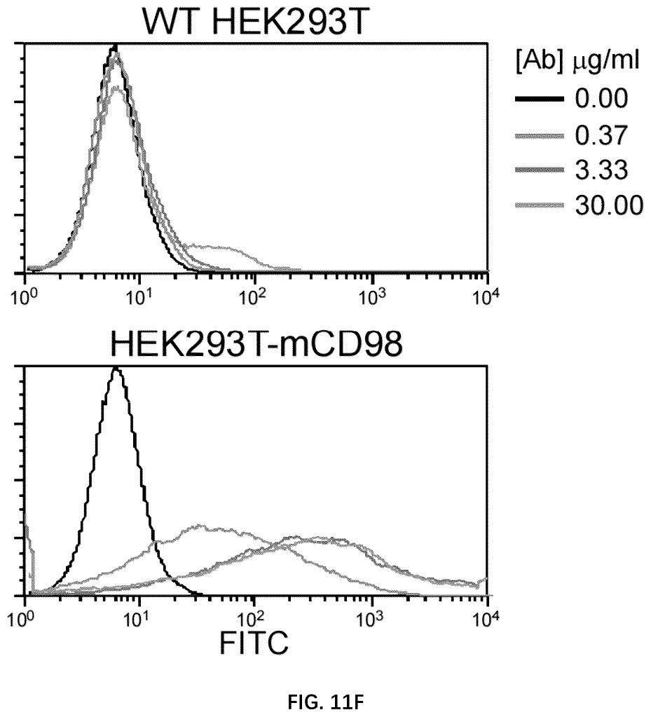

To solve this problem, a model of CD98 humanized mice were generated by CRISPR/Cas9 to replace mCD98 ECD with its human counterpart in a C57BL/6 genetic background (using C57BL/6 mice as a recipient) (FIG. 11A-D). sgRNA (sgRNA-CD98: ccgcgctgccgtgagctgcctgt; SEQ ID NO:1) targeting sequences within the first intron was used for germ line integration of the sequence of hCD98 ECD (aa 107-529) into the Slc3a2 locus of C57BL/6 mice, resulting in the elimination of the mouse Slc3a2 gene. As a template for homology-directed repair (HDR), the targeting vector was constructed harboring a 1272 bp sequence encoding aa 107-529 of hCD98, a polyA element, an 829 bp upstream fragment and a 945 bp downstream fragment extending from the sgRNA-CD98 targeting site (FIG. 11A). From pronuclear microinjections of Cas9 protein, sgRNA-CD98, and the targeting vector into C57BL/6 zygotes, 2 live pups (F0) were obtained. The correct insertion of hCD98 ECD in the two FO mice was verified by multiple pairs of primers (FIGS. 11A-D, Table 2). Heterozygous F0 mice were crossed with wild type C57BL/6 mice to obtain F1 generation mice. Heterozygous F1 animals were then interbred to produce homozygous hCD98 knock-in mice (named as CD98 humanized mice).

TABLE 2
PCR primers used in CD98 humanized mice identification

| | | Sequence | SEQ ID | PCR product size (bp) | |
|---|---|---|---|---|---|
| PCR type | Primers | (5′ > 3′) | NO: | Knock-in allele | WT allele |
| CD98 | Y1-F | GGGTCAAGTTCACCGGCTTA | 2 | 283 | / |
| humanized | Y1-R | CTCAGGTAATCGAGACGCCC | 3 | | |
| transgene | | | | | |
| WT allele | WT1-F | GTGAAGATCAAGGTGGCGGA | 4 | 1630 | 364 |
| | WT1-R | AAAAAGCAGCAGAATGCCCG | 5 | | |
| Correct | MS2-F | GGGTCAAGTTCACCGGCTTA | 6 | 2219 | 825 |
| integration | MS2-R | CTGGGCCAGAATGCCAAAC | 7 | | |
| sequence | | | | | |
| Correct | IF-1 | GTGTAAGCCCCGAGGAGAAT | 8 | 996 | / |
| integration | Y1-R | CTCAGGTAATCGAGACGCCC | 3 | | |
| site | | | | | |

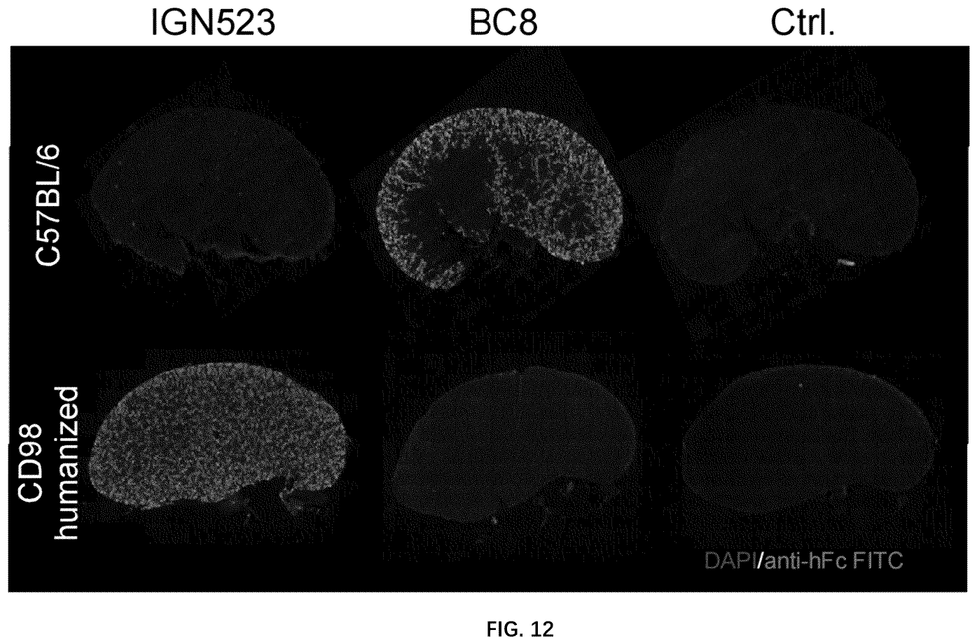

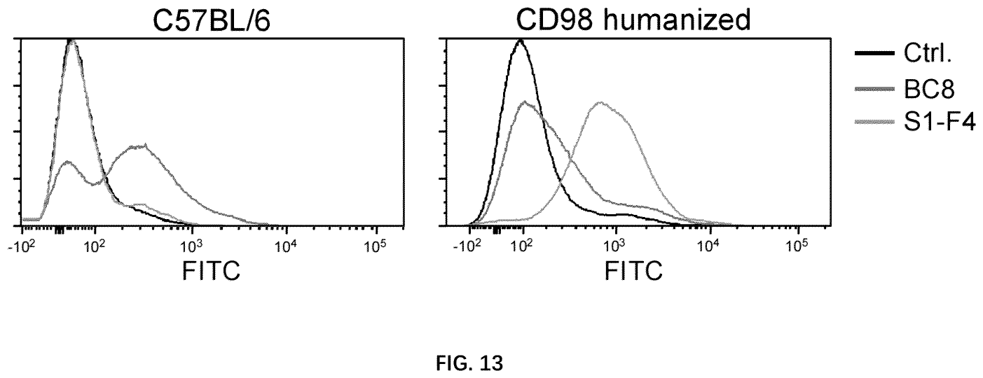

The CD98 humanized mouse model as generated retained the physiological function of mCD98, which enables testing of S1-F4 in a model with CD98 expression in normal tissues and with intact immune systems. The expression profile of hCD98 in different tissues of the established CD98 humanized mice was examined by immunofluorescence staining assays, confirming similar hCD98 expression pattern in tissues as mCD98 expression in wild type mice (FIGS. 12-13).

### Example 4. Antitumor Activity of S1-F4 Against Aggressive and Refractory Tumors

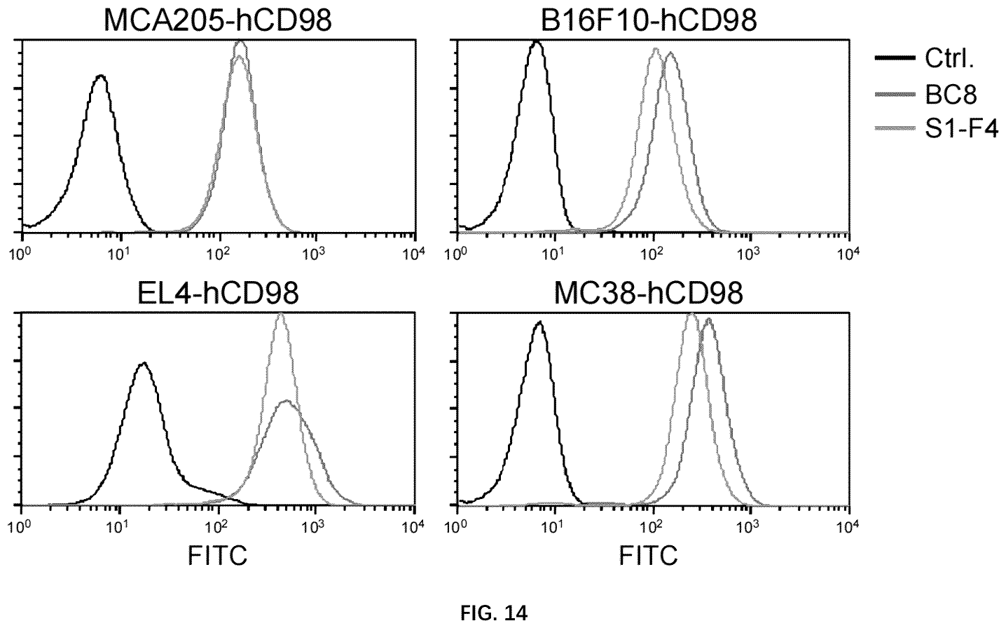

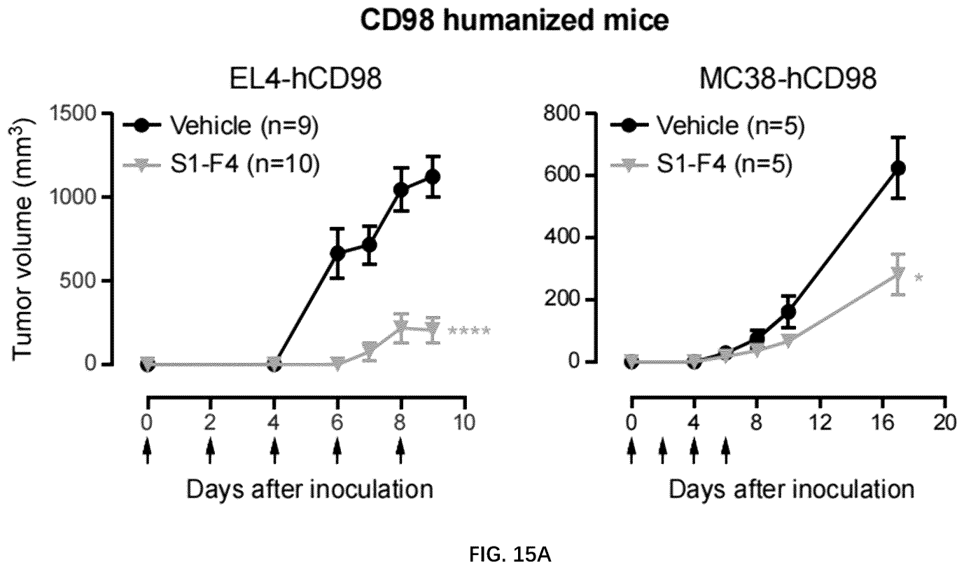

The CD98 humanized mice was used to evaluate the antitumor activity of S1-F4 in four aggressive and difficult-to-treat mouse tumor models. The models were established using mouse tumor cell lines stably expressing hCD98 (FIG. 14).

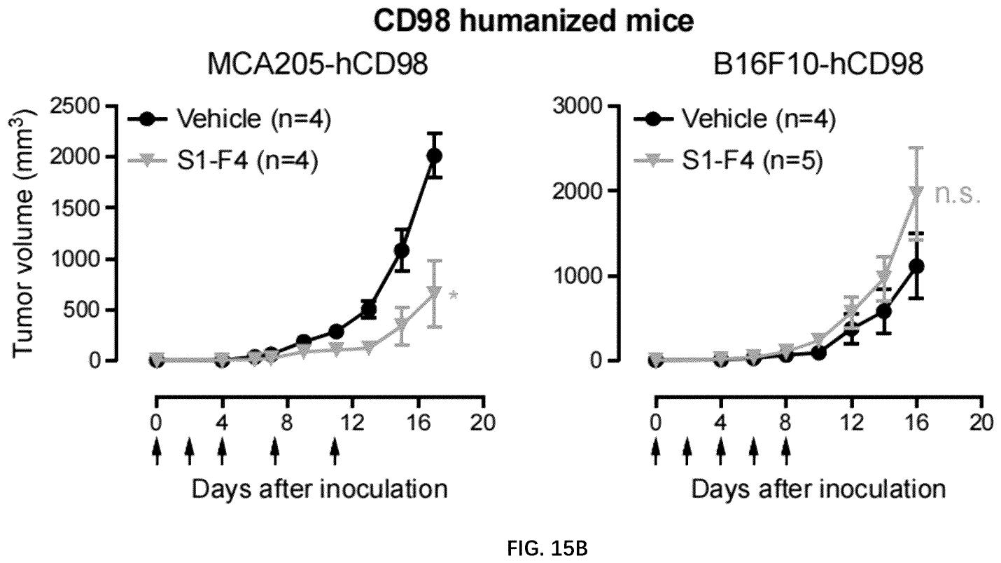

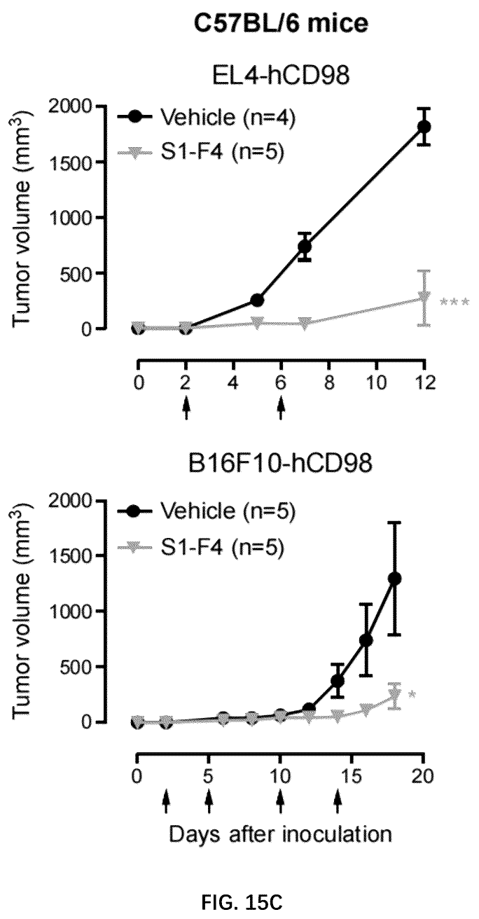

S1-F4 treatment exhibited significant antitumor effects against three of the four tumor models: EL4-hCD98, MC38-hCD98, and MCA205-hCD98; it exerted no obvious effect against B16F10-hCD98 tumors (FIG. 15A). These results demonstrate that S1-F4 exerts potent and broad-spectrum antitumor activity against multiple malignancies in immune-deficient xenograft and immune-competent syngeneic mouse models.

Notably, when testing S1-F4 treatment against EL4-hCD98 and B16F10-hCD98 tumors in wild type C57BL/6 mice (i.e., in mice without any hCD98 expression in non-tumor tissues), S1-F4 treatment induced significant suppression of tumor growth in both tumor models (FIG. 15B). S1-F4 exhibited antitumor effects against B16F10-hCD98 in wild type mice but no such effect in the CD98 humanized mice, suggesting that the lack of “antigen sink” in wild type mice may contribute to the antitumor effects of S1-F4 in this highly aggressive tumor model. By “antigen sink” it means that an antigen-mediated clearance of antibodies targeting cell membrane antigens. These results indicated that antitumor effects of an anti-CD98 antibody observed in wild-type mice with human xenografts do not always constitute a reasonable expectation of their antitumor effects in human use.

### Example 5. Antitumor Activity of S1-F4 Depends on Fc-FcγR Interactions

This example investigates whether the antitumor activity of anti-hCD98 antibodies depends on Fc-mediated immune responses, for example complement dependent cytotoxicity (CDC), antibody dependent cell-mediated cytotoxicity (ADCC), or phagocytosis (ADCP).

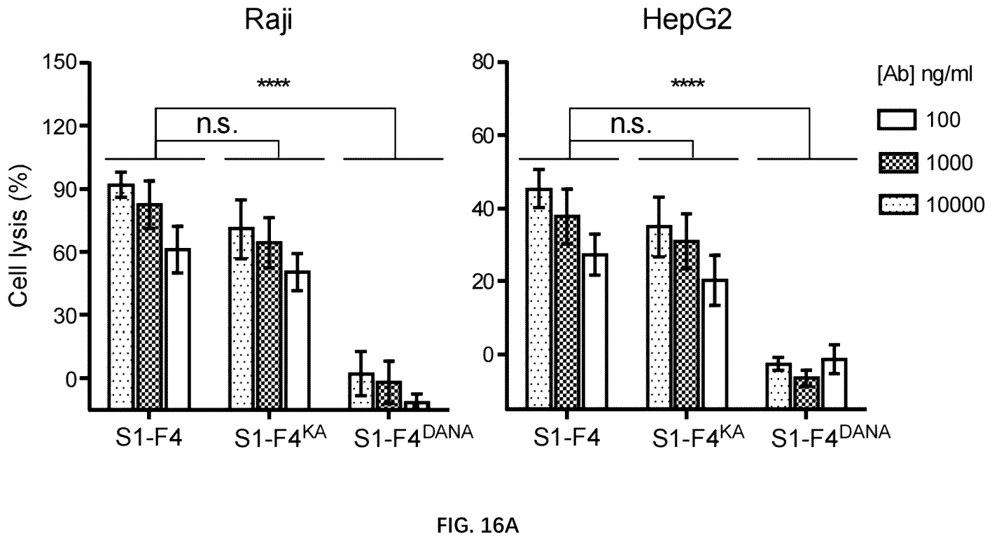

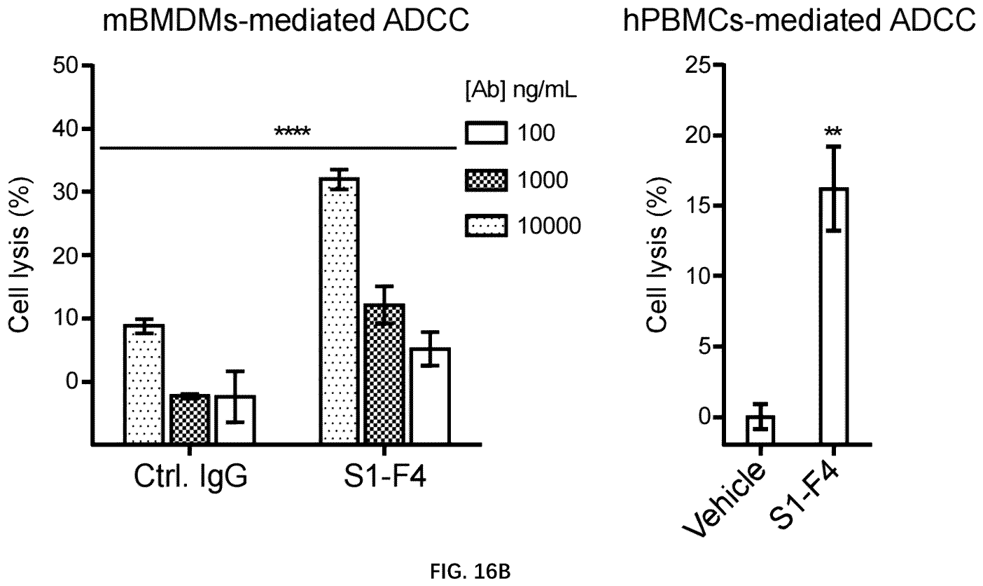

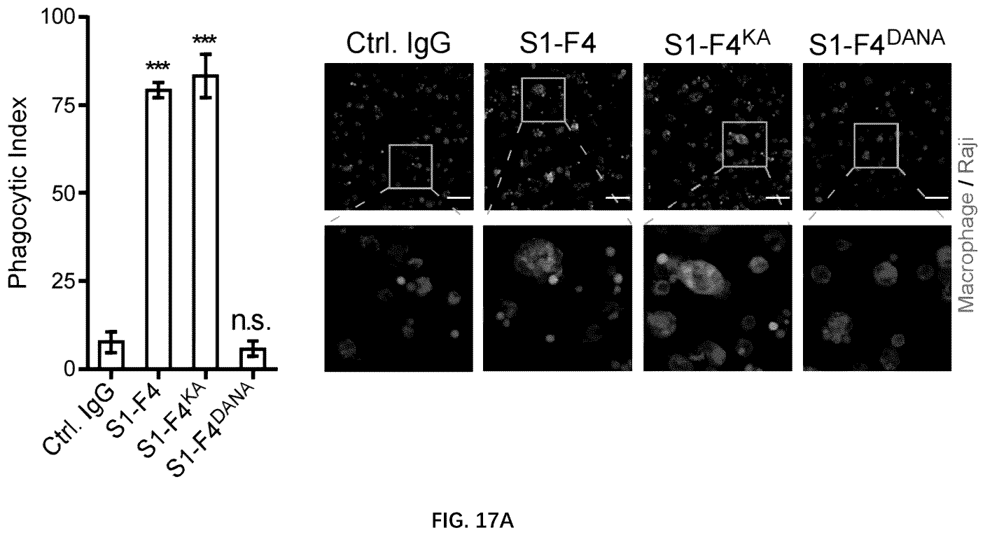

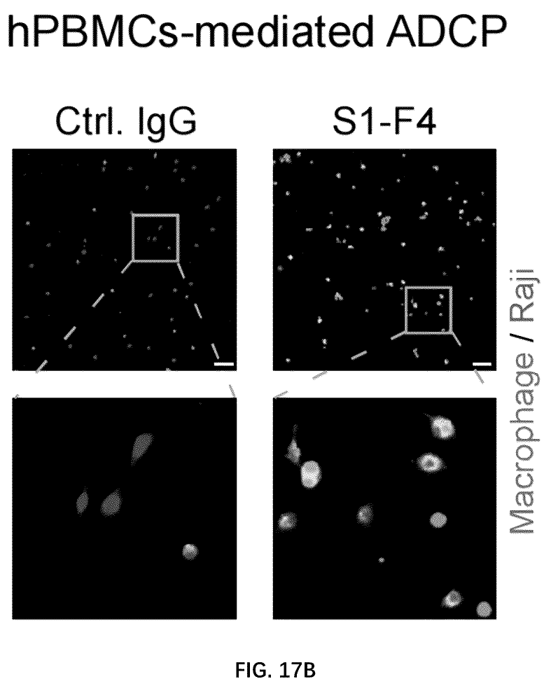

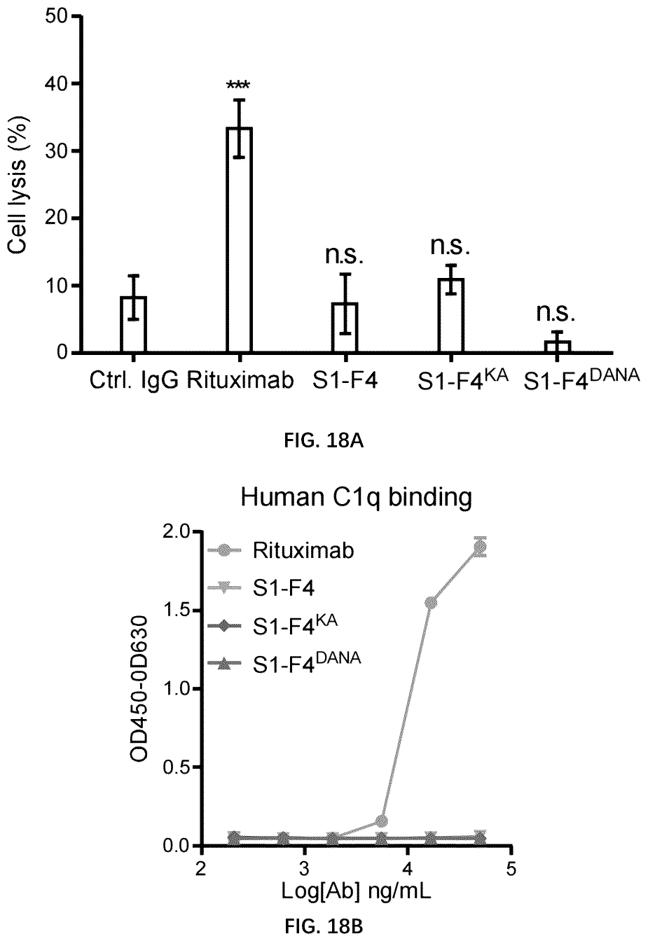

To dissect Fc-related functions of S1-F4, two S1-F4 variants, S1-F4^{KA} and S1-F4^{DANA}, were generated (FIG. 3). S1-F4^{KA} bears a K322A (KA) mutation to eliminate binding of Fc with C1q, resulting in abrogation of CDC. S1-F4^{DANA} harbors D265A/N297A (DANA) mutations that abolish Fc binding to FcγRs, leading to abrogation of ADCC and ADCP. The S1-F4 and its two variants' ability in mediating ADCC, ADCP, and CDC was tested. In vitro assays showed that S1-F4 and S1-F4^{KA} induced ADCC (FIG. 16A, FIG. 16B) and ADCP killing (FIG. 17A, FIG. 17B) against Raji and/or HepG2 cells, whereas the S1-F4^{DANA} variant did not. Neither S1-F4 nor the tested mutant variants were able to bind to human C1q or elicit CDC killing against Raji cells (FIG. 18A, FIG. 18B).

These results indicate that S1-F4 could bind to FcγRs and induce ADCC and ADCP killing, while could not bind to C1q or induce CDC killing.

### Example 6. Effect of S1-F4 on CD98's Function

This example investigates if S1-F4 interferes with CD98's biochemical or cell biological functions in the tumor cell lines earlier found to be highly sensitive to S1-F4 treatment.

Amino acid uptake assay was conducted to analyze the effect of S1-F4 in amino acid transport. L-Homopropargylglycine (HPG), a methionine mimic, was used as a traceable substrate for the LAT1 (L-type/large neutral amino acid transporter 1)/CD98 heterodimer HAT complex, and the LAT1/CD98 complex inhibitor BCH (2-amino-2-norbornane-carboxylic acid) was used as a positive control for disrupting transport function.

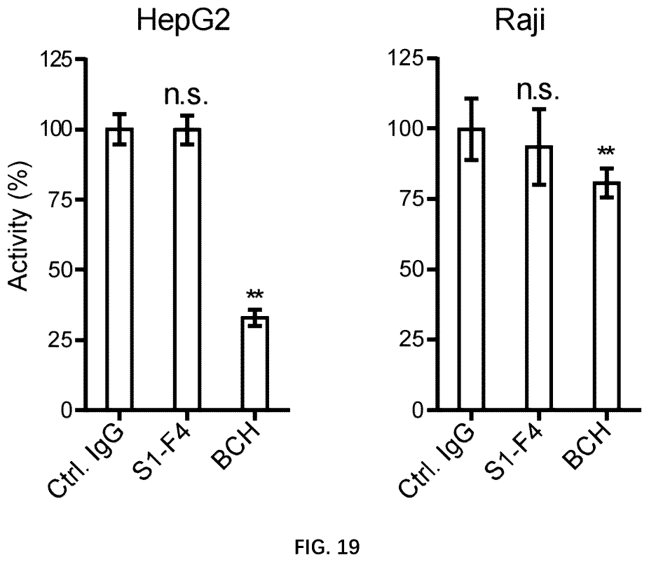

As shown by FIG. 19 comparing HPG uptake efficiency of the control IgG, S1-F4, and BCH-treated cells, only BCH impaired HAT complex transport activity. These results show that S1-F4 does not affect amino acid transport.

Since CD98 increases cell proliferation through enhancement of integrin signaling, S1-F4 was tested in cell proliferation assays for its inhibitory effects on the growth of HepG2, HCT-8, Raji, and Ramos cells (four of the highly S1-F4 sensitive lines).

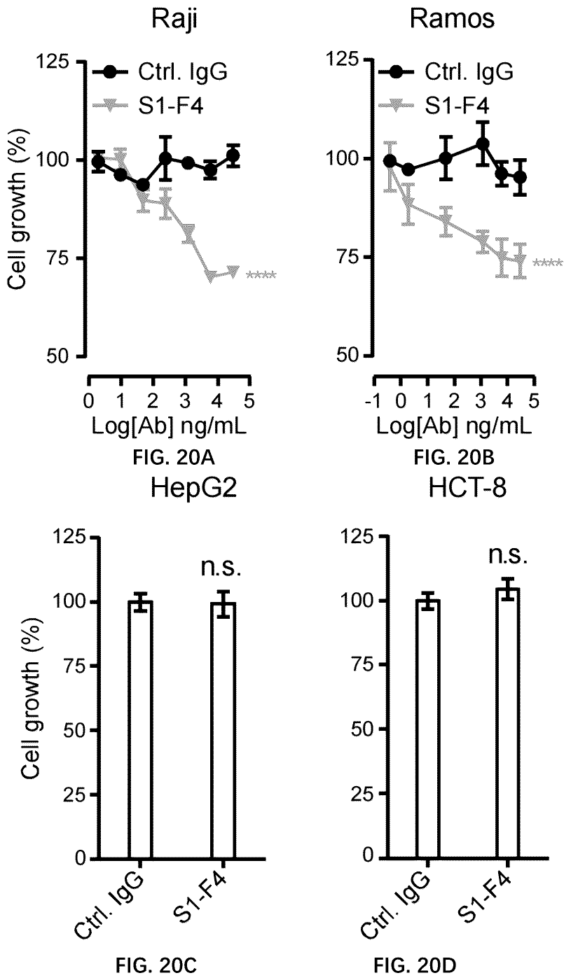

As shown in FIGS. 20A-20D, S1-F4 exhibited cell growth inhibition on Raji and Ramos cells in a dose-dependent manner, but no S1-F4-mediated effect on growth was observed for HepG2 or HCT-8 cells. These results show that cell growth inhibition is not required for S1-F4's antitumor activity.

The inventors also interrogated S1-F4's mechanism(s) of action (MOA) by comparing the antitumor efficacy of S1-F4 and its two Fc variants in both xenograft and syngeneic tumor models. For xenograft tumor models, HepG2 and Raji tumors were selected to represent resistant and sensitive cells for S1-F4's inhibition of cell growth, respectively. For the syngeneic tumor models, EL4-hCD98 and MC38-hCD98 cells were selected to establish tumors in CD98 humanized mice.

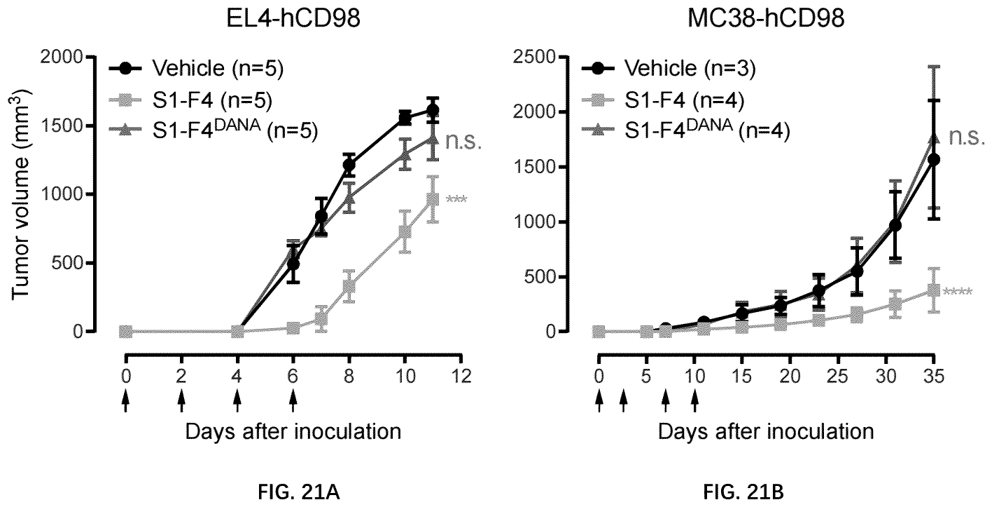

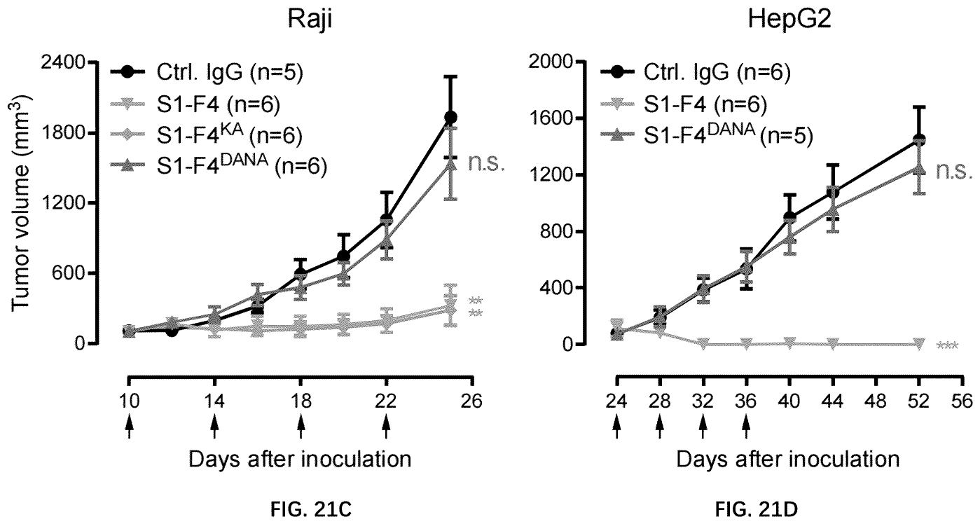

S1-F4^{DANA} exerted no therapeutic effect in any of the four models (FIG. 21), supporting that S1-F4's antitumor activity depends on Fc-FcγR interactions. In addition, in the Raji xenograft tumor model, S1-F4 and S1-F4^{KA} elicited similarly potent inhibition of tumor growth (FIGS. 21A-21D, lower left panel), confirming that CDC function is not required for antitumor activity of S1-F4. Collectively, these results demonstrate that the antitumor activity of S1-F4 depends on both ADCC and ADCP mediated via Fc-FcγR interactions.

### Example 7. Involvement of Immune Cells in the Antitumor Activity of S1-F4

To characterize which immune cell subset(s) participate in S1-F4's antitumor effects, experiments were conducted with HepG2 tumor models, in which subset-specific antibodies were used to deplete macrophages, NK cells, or neutrophils prior to and optionally during S1-F4 treatment. It was found that macrophage depletion abrogated S1-F4's antitumor activity, whereas S1-F4 still exerted potent antitumor activity upon neutrophil depletion and NK depletion. It can be concluded that macrophages are required for S1-F4's antitumor efficacy in the HepG2 xenograft model.

Recalling that two (A549, A-431) xenograft models were resistant to S1-F4 treatment, the impact of S1-F4 treatment on macrophage infiltration was examined. It was found that S1-F4 treatment significantly increased the proportion of macrophages in the HepG2 tumors by over 2-folds. In contrast, S1-F4 treatment did not increase the number of infiltrated macrophages in S1-F4-resistant A-431 tumors or HCT 116 tumors. These results indicate that the lack of intratumoral macrophage infiltration may contribute to tumor cell resistance to S1-F4 treatment.

In addition to the immune-deficient xenograft tumor models, the effect of depleting macrophages, NK cells, or neutrophils on S1-F4's antitumor efficacy was also evaluated in the EL4-hCD98 syngeneic tumor model established with the CD98 humanized mice. Consistent with the results obtained in the HepG2 tumor model, only macrophage depletion abrogated the therapeutic effects of S1-F4; neither neutrophil depletion nor NK depletion altered S1-F4's antitumor activity. Thus, macrophages are essential for S1-F4's antitumor effects in both xenograft and syngeneic tumor models.

CD98 humanized mice have an intact immune system, thus providing a suitable model system to study the role of T cell immune responses in S1-F4's antitumor efficacy. To test whether S1-F4 is able to mobilize T cells to attack tumor cells, the EL4-hCD98 tumor model established in CD98 humanized mice was employed to evaluate the impacts of depleting CD4+ T cells or CD8+ T cells on S1-F4's antitumor activity. As a result, depleting CD4+ T cells did not alter the antitumor activity of S1-F4, indicating that CD4+ T cells apparently do not participate in S1-F4's antitumor effects. Depleting CD8+ T cells significantly attenuated S1-F4's inhibition of EL4-hCD98 tumor growth. Thus, CD8+ T cells participate in S1-F4's antitumor activity, and macrophages alone are insufficient to induce long-term antitumor effects.

The inventors also investigated whether dendritic cells (DCs) function as APCs that cross-prime CD8+ T cells during S1-F4 treatment. Specifically, CD11c-DTR (diphtheria toxin receptor) (Itgax-DTR/EGFP) mice bearing EL4-hCD98 tumors were treated with diphtheria toxin (DT) to deplete DCs before (and during) S1-F4 treatment. Depleting DCs cells significantly impaired S1-F4's inhibition of EL4-hCD98 tumor growth. Thus, DCs are essential for the full S1-F4-induced antitumor response.

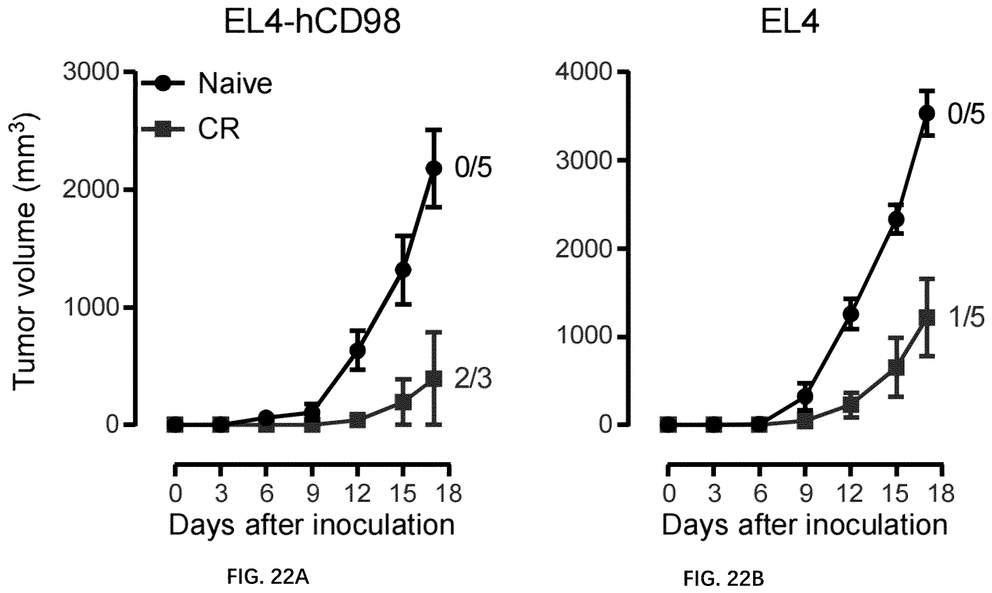

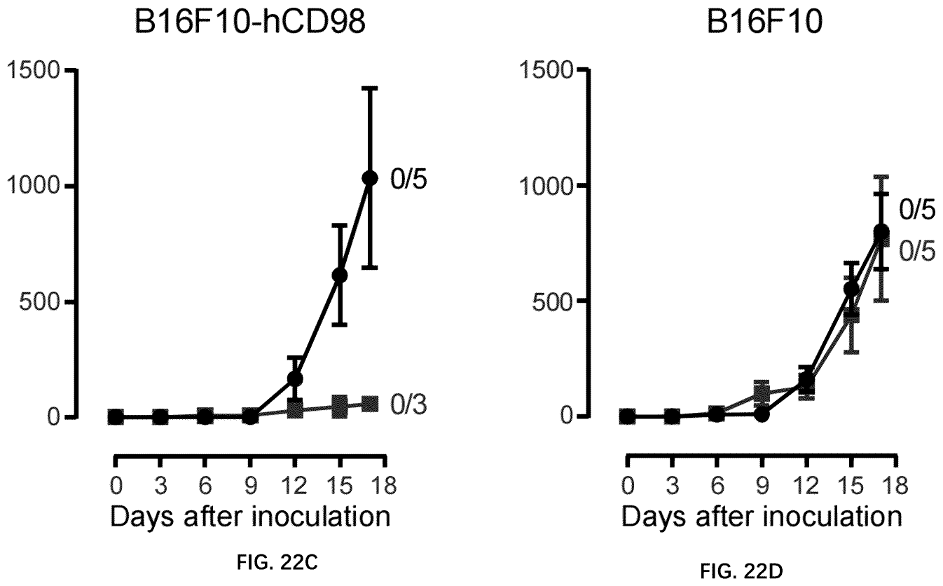

Finally, it was found that the C57BL/6 mice displaying a complete response (CR) against EL4-hCD98 tumors upon S1-F4 treatment were protected from subsequent challenge with the same tumor cells, EL4-hCD98, or with EL4, B16F10 or B16F10-hCD98 tumor cells, exhibiting either a significant delay in tumor growth or complete tumor rejection, indicating an effect of S1-F4 in preventing recurrence of tumor (FIGS. 22A-22D). As expected, the CR mice were not able to survive the challenge with B16F10 tumor cells (FIG. 22) that unlikely share common antigens with EL4-hCD98, supporting that S1-F4 treatment can induce antigen-specific antitumor immune memory.

Considering these results together, the inventors propose that the MOA of S1-F4 comprises the following steps: 1) S1-F4 induces ADCC and ADCP to attack CD98-expressing tumor cells via its Fc engagement with FcγRs on macrophages; 2) tumor cell death resulted from step 1) produces tumor cell associated antigens that are subsequently cross-presented to CD8+ T cells via DCs; 3) CD8+ T cells are activated to attack tumor cells. During this process, macrophages exert a function in initiating antitumor responses, while cytotoxic CD8+ T cells are induced after the initiation of antitumor responses, specifically resulting from the increasing cross-presentation mediated by DCs. Thus, S1-F4 treatment may bridge the innate immune system and the adaptive T cell immune system to attack tumor cells and to induce long-term immune memory.

### Example 8. Generation of pH-Dependent Anti-hCD98 Antibodies

The inventors evaluated the safety and pharmacokinetic (PK) properties of S1-F4 in cynomolgus monkeys. Two doses of S1-F4 treatment (20 mg/kg at day 0 and 10 mg/kg at day 15) caused no obvious side effects, suggesting a desirable safety of S1-F4 at therapeutically effective doses.

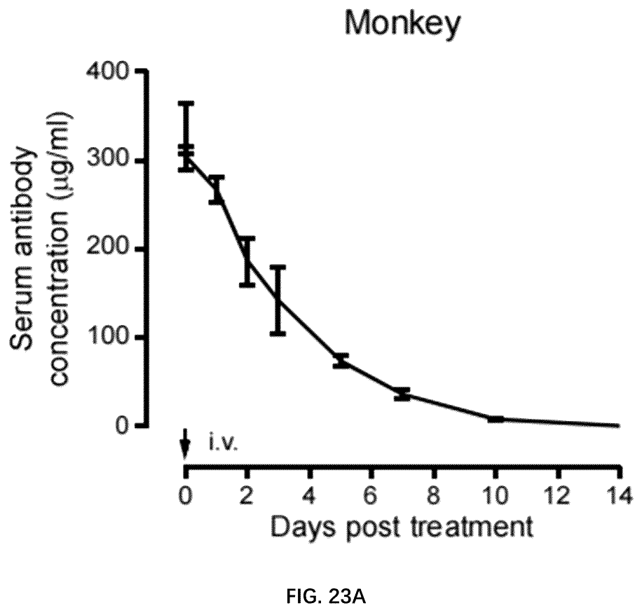

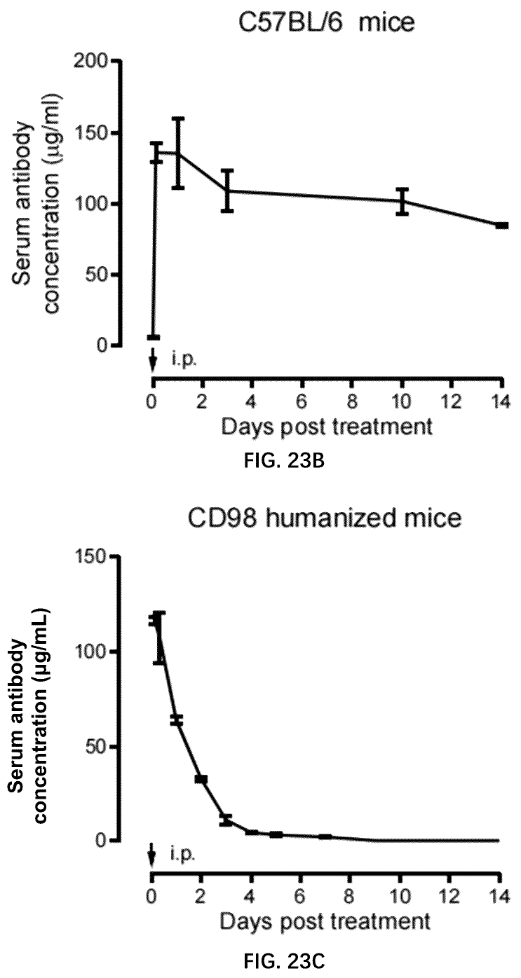

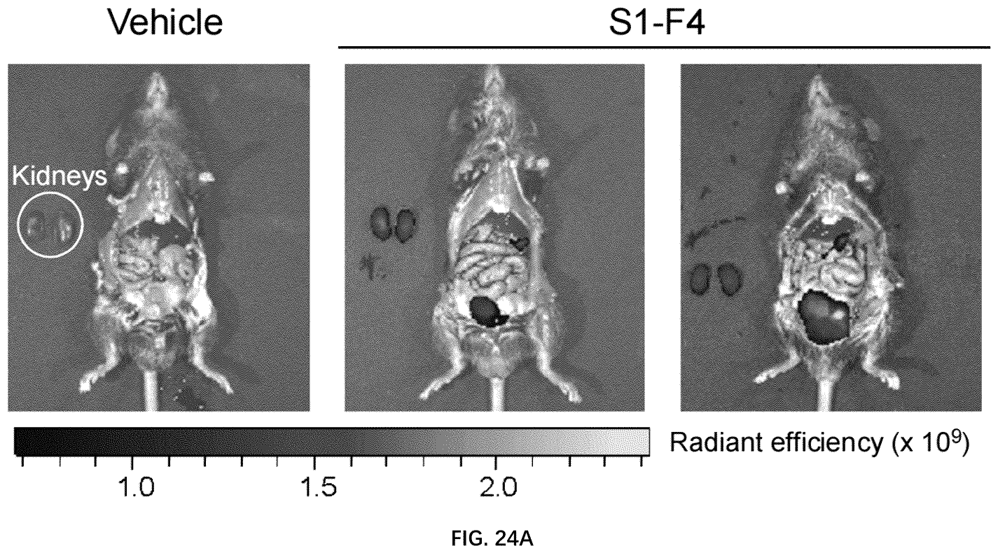

However, the S1-F4 serum concentration decreased rapidly in the monkeys over time (FIGS. 23A-23B), with a short half-life (t½ ˜29.5 hr). S1-F4's serum concentration was also measured in C57BL/6 and CD98 humanized mice after a single-dose administration. It was found that it decreased rapidly in CD98 humanized mice (t½ ˜69.6 hr), while decreased slowly in C57BL/6 mice (t½ ˜341.5 hr) (FIG. 23C).

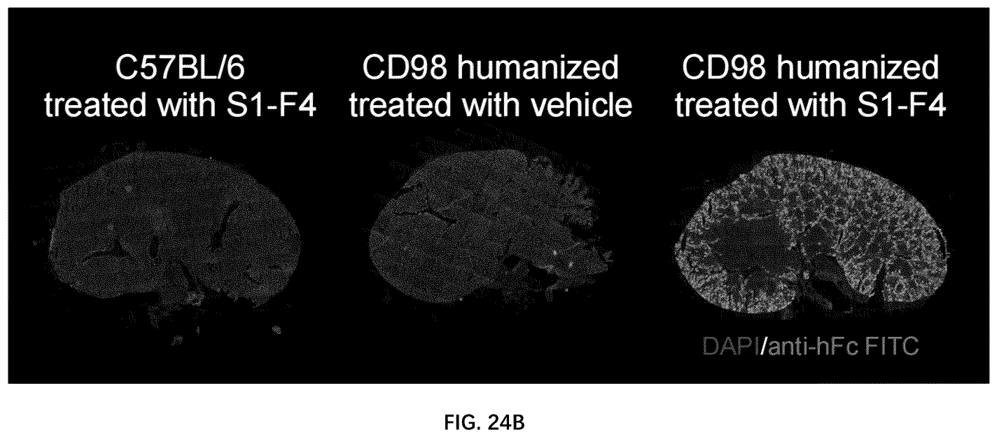

Consistent with the strong expression of murine CD98 in kidneys, strong fluorescence signals in the kidneys of CD98 humanized mice was observed after administration of fluorescent-labeled S1-F4 (S1-F4-Cy7) (FIG. 24A). Immunofluorescence staining also revealed strong S1-F4 enrichment in kidneys of CD98 humanized mice but not in C57BL/6 mice after treatment with S1-F4 (FIG. 24B). It is conceivable that the rapid clearance of S1-F4 in cynomolgus monkeys and CD98 humanized mice results from an “antigen sink” phenomenon—S1-F4 binding to the CD98 proteins present in normal tissues.

Seeking to overcome the impacts of the antigen sink effect and to improve PK profile, the inventors creatively turned to engineer S1-F4 into a pH-dependent antibody that will bind strongly to CD98 under acidic conditions (pH 6.5-6.9) such as in tumor microenvironment but bind weakly (or not at all) under neutral (pH 7.2-7.5) conditions in most normal tissues. An ideal pH-dependent antibody would retain S1-F4 antitumor activity while exhibiting preferential binding to antigens in solid tumors.

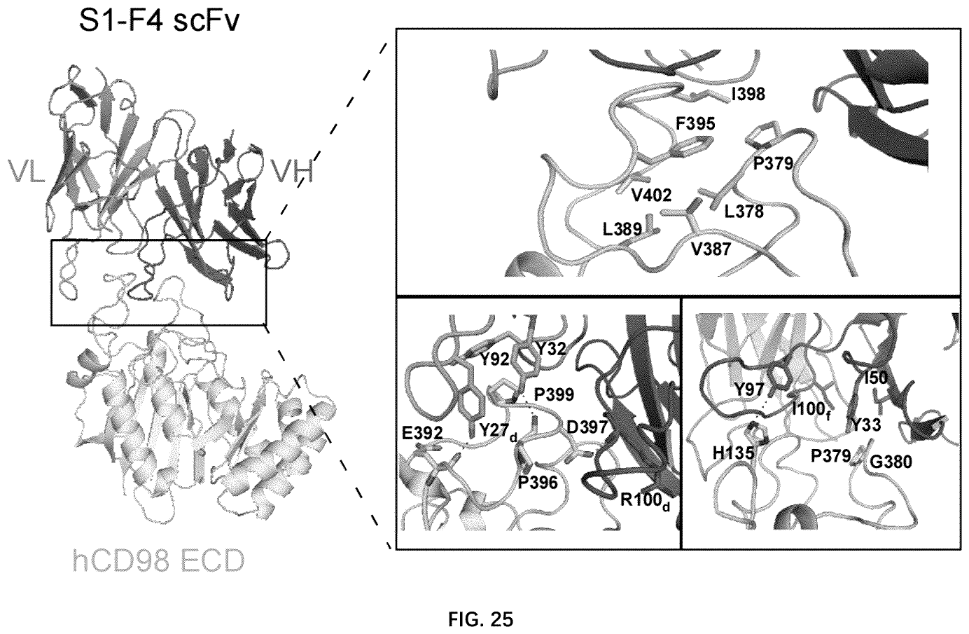

To support the rational engineering of S1-F4, the crystal structure of the hCD98 ECD in complex with S1-F4 was solved to gain accurate information about the binding interface (FIG. 25). The crystal structure of S1-F4 scFv-hCD98 ECD complex was solved at 2.8 Å resolution, revealing that S1-F4 recognizes a conformational epitope on hCD98 ECD which comprises residues 376-384 and 391-399 from two loop regions (FIG. 24). The structure showed that 7 hCD98 ECD residues (L378, P379, V387, L389, F395, I398, and V402) form a hydrophobic core that stabilizes the conformation of the two loops that contribute to the conformational epitope (FIG. 25).

Assessing the specific architecture of the epitope engaging S1-F4's VH and variable light (VL) chains showed that hCD98 ECD residues 391-399 engage with S1-F4 LCDR1, LCDR3, and HCDR3. The side-chain hydroxyl groups of the S1-F4 LCDR1 Y27d and Y32 residues respectively form hydrogen bonds with main chain amide groups of CD98 ECD residues E392 and P396. Further, the aromatic side-chains of S1-F4 LCDR1 Y32 and LCDR3 Y92 clamp CD98 ECD's P399 residue through non-polar interactions (FIG. 25). The side chain of CD98 ECD's D397 forms a hydrogen bond with the main-chain of R100d in the S1-F4 HCDR3 region. Also, the S1-F4 VH mediates the antibody's interactions with CD98 ECD residues 376-384. A kink in this loop formed by P379 and G380 fits tightly into a hydrophobic groove comprising S1-F4 VH residues Y33, I50, and I100f on HCDR1, HCDR2, and HCDR3 respectively. Near this epitope, the side chain of CD98 ECD H135 is hydrogen-bonded to S1-F4 HCDR3 Y97 (FIG. 25).

Supporting that this conformational epitope does mediate S1-F4 scFv-hCD98 ECD complex binding, FACS-based binding analysis showed that alanine substitution of two amino acids positioned at the binding interface (L389A and P399A) markedly reduced the binding of S1-F4 to hCD98. L378A, F395A and D397A mutations also attenuated binding, supporting a stabilizing influence from these hydrophobic core residues on the affinity of S1-F4 scFv-hCD98 ECD binding. It was notable that none of these alanine substitution mutations affected the binding of IGN523 to hCD98, indicating that S1-F4 and IGN523 bind to distinct epitopes.

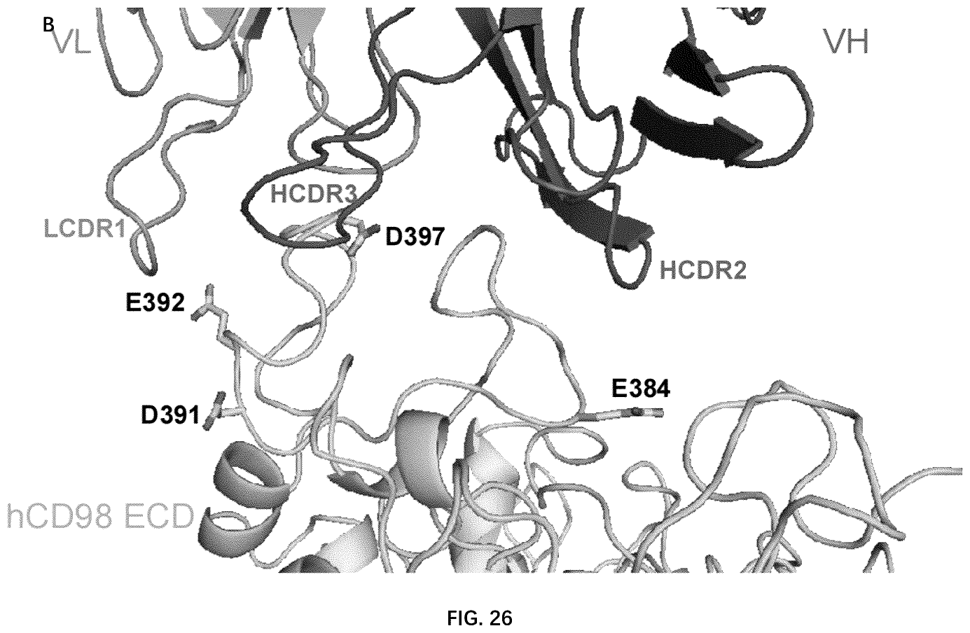

Previous studies have shown that protonation of ionizable residues like histidine (H) can contribute to pH-dependent binding (Chaparro-Riggers et al., 2012; Johnston et al., 2019; Sarkar et al., 2002). The inventors therefore hypothesized that increasing the extent of interactions between H and acidic amino acids (aspartate (D) or glutamate (E)) may promote pH-dependent binding between S1-F4 and CD98. The structure indicates that four acidic residues (E384, D391, E392, and D397) are within S1-F4's epitope on hCD98 ECD (FIG. 26), and H135 is located near the epitope, forming close contact with S1-F4 HCDR3 Y97 (FIG. 25), thereby providing a molecular basis for the rational engineering of pH-dependent S1-F4 variant antibodies.

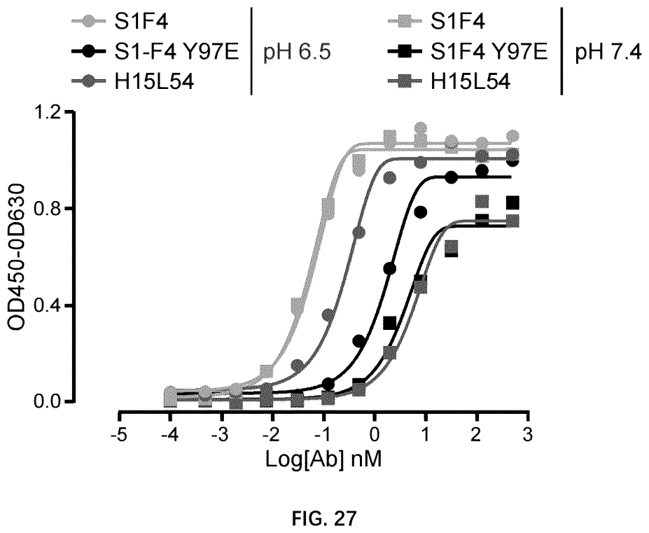

First, Y97E mutation was introduced on S1-F4 HCDR3 to generate S1-F4 Y97E. In ELISA assay, the binding activity of S1-F4 for CD98 ECD was comparable at pH 6.5 (EC50=0.059 nM) and pH 7.4 (EC50=0.046 nM). In contrast, the binding of S1-F4 Y97E to CD98 at pH 6.5 (EC50=1.686 nM) was significantly stronger than at 7.4 (EC50=4.527 nM) (FIG. 27), validating that changing a residue in close contact with the acidic residues in the epitope to an acidic residue is an effective strategy to obtain an antibody variant with pH-dependent antigen binding.

Further, to obtain anti-CD98 antibodies with improved binding activity and pH-dependence, the inventors screened phage display sub-libraries derived from S1-F4 VH Y97E reflecting mutations at positions facing four acidic residues of CD98: HCDR3—facing D397, HCDR2—facing E384, and LCDR1—facing both D391 and E392 (FIG. 26). H15L54, another pH-dependent anti-hCD98 antibody, was identified.

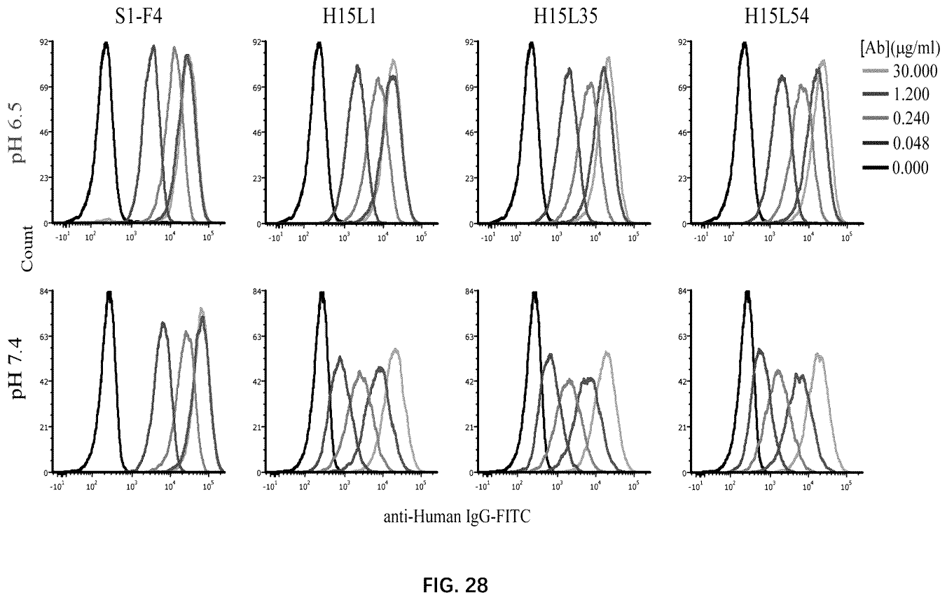

The binding activity of H15L54 at pH 6.5 (EC50=0.247 nM) was about 7 times higher than S1-F4 Y97E (EC50=1.686 nM), and its binding pH-dependence (EC50 ratio, pH 7.4/6.5=38.0) was about 14 times higher than S1-F4 Y97E (EC50 ratio, pH 7.4/6.5=2.7) (FIG. 27). FACS indicated no obvious difference in S1-F4 binding to A-431 cells at pH 6.5 or pH 7.4, whereas H15L54 binding to A-431 cells was significantly stronger at pH 6.5 than at pH 7.4 (FIG. 28).

A modeled structure manually built by PyMOL indicated that 4 pairs of H-D/E interactions formed between H15L54 and CD98 after S1-F4 engineering: H15L54 VH H54, VH E97, VH H100_{d}, and VL H27_{f} respectively interact with CD98 ECD E384, H135, D397, and E392. These four pairs of H-D/E interaction likely contribute to the highly selective low-pH binding between H15L54 and CD98.

### Example 9. Preferential Binding and Improved Antitumor Activity of the pH-Dependent Anti-hCD98 Antibody Against CD98 in Tumors

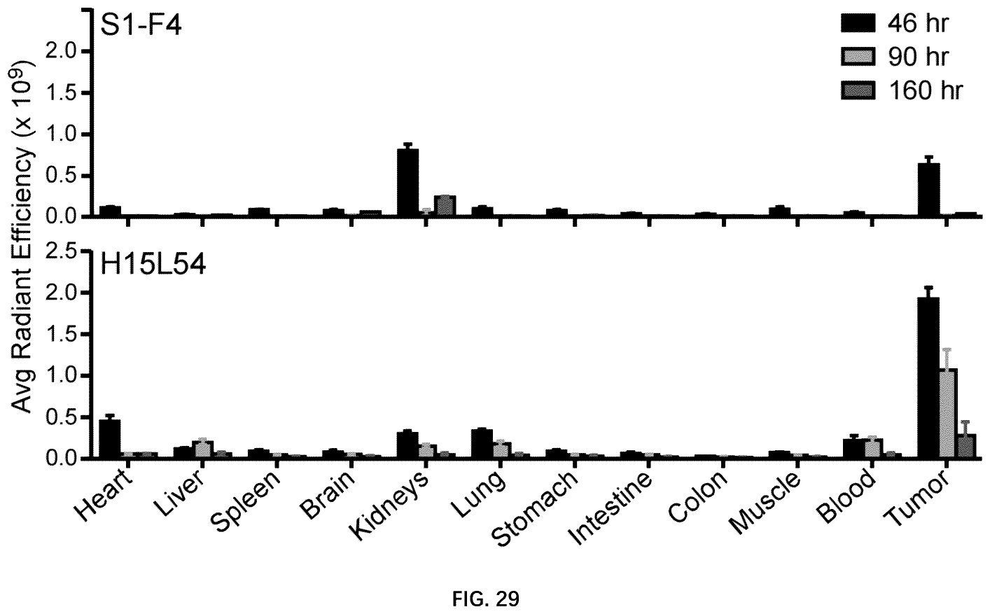

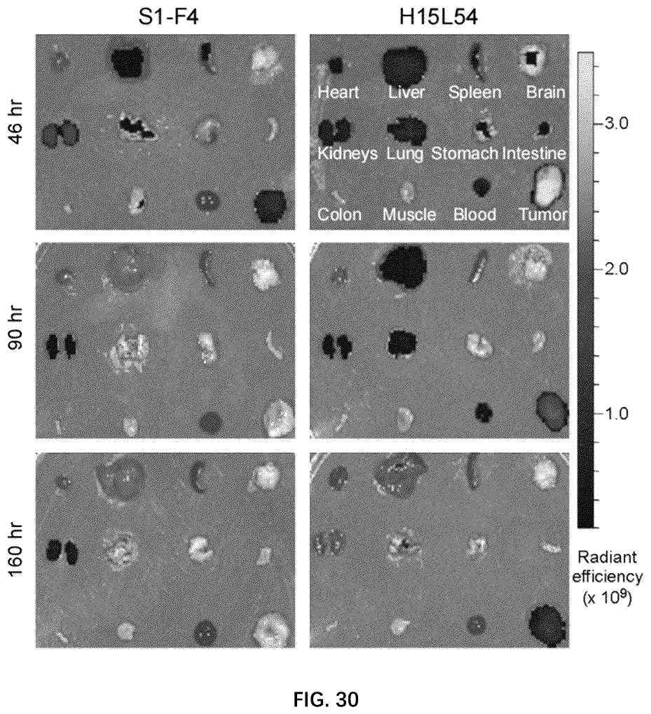

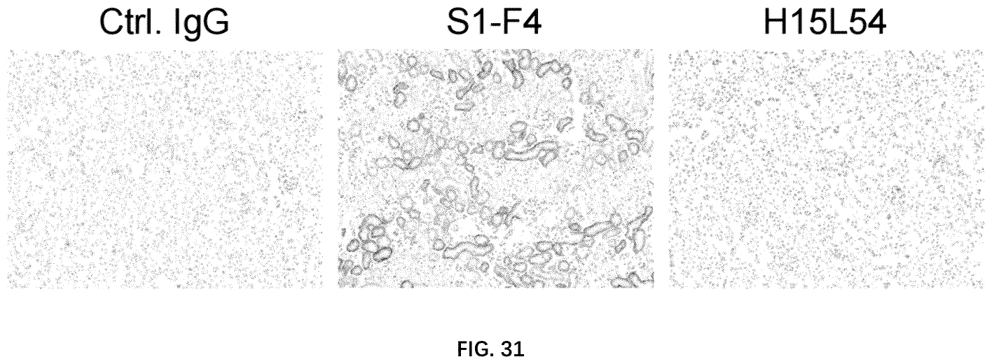

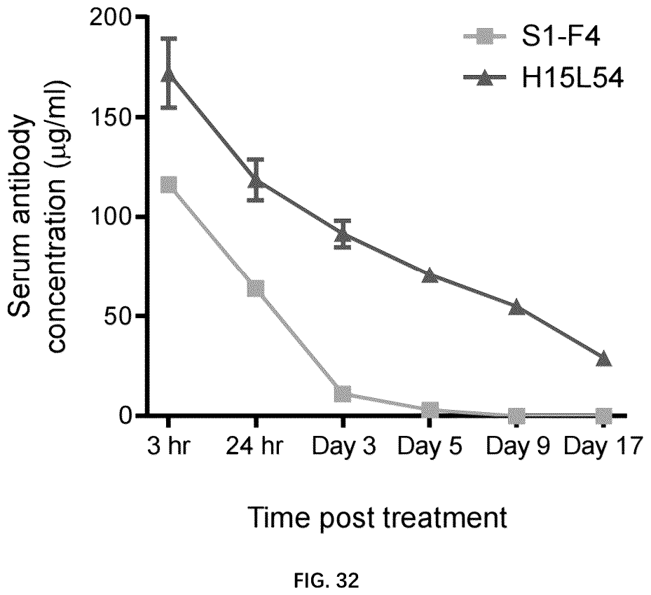

The distribution of antibody was studied by administering fluorescently labeled H15L54-Cy7 antibodies to CD98 humanized mice bearing EL4-hCD98 tumors. In sharp contrast to the accumulation of S1-F4-Cy7 in kidneys, H15L54-Cy7 accumulated primarily to tumors (FIGS. 29-30). Then immunofluorescence staining was used to detect enrichment of the S1-F4 and H15L54 antibodies in kidneys of CD98 humanized mice. Consistent with previous findings, mice treated with S1-F4 had a strong FITC signal in their kidneys, whereas only a very weak FITC signal was evident in kidneys of mice treated with H15L54 or control IgG (FIG. 31). A comparison of the PK properties of S1-F4 and H15L54 in the CD98 humanized mice showed that the serum concentration of H15L54 (t½ ˜217.1 hr) decreased much more slowly than S1-F4 (t½ ˜69.6 hr) (FIG. 32). These results support that, the pH-dependent-binding property of H15L54 enables its preferential binding to CD98 in tumors over normal tissues at physiological pH in vivo.

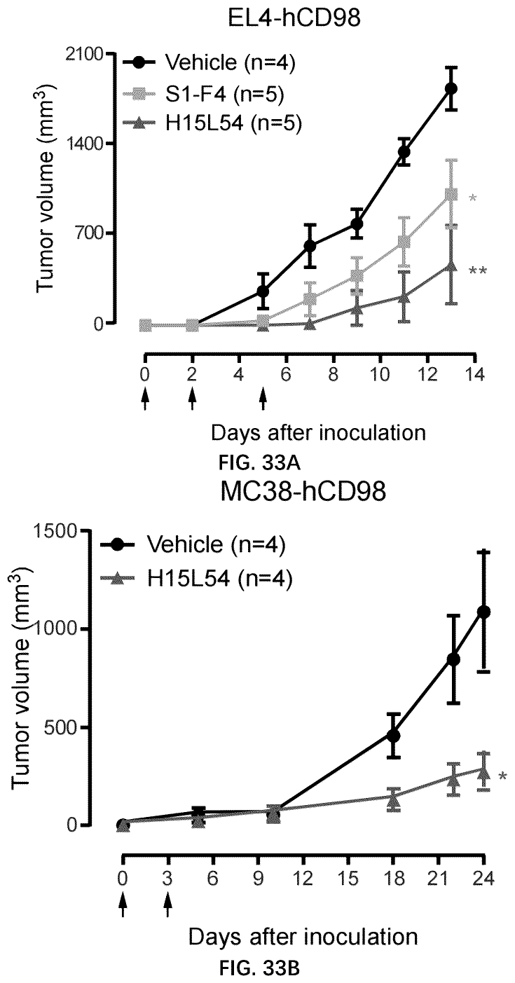

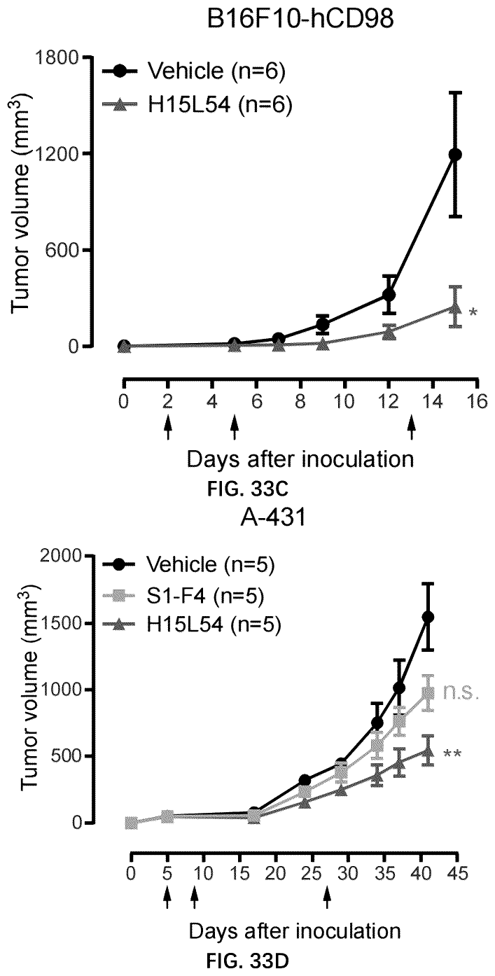

The antitumor activity of H15L54 was evaluated in multiple tumor models. In EL4-hCD98 and MC38-hCD98 tumor models (both are sensitive to S1-F4 treatment) established in the CD98 humanized mice, H15L54 exhibited significant antitumor activity (FIGS. 33A-B). In addition, H15L54 showed significant antitumor activity in tumor models resistant to S1-F4 treatment, including B16F10-hCD98 tumor models established in CD98 humanized mice and A-431 xenograft tumor models (FIGS. 33C-D). Thus, the pH-dependent anti-hCD98 antibody H15L54 confers improved antitumor activity and desirable PK profile by eluding the likely “antigen sink” problem of S1-F4 via its preferential antigen binding in the relatively low pH conditions of the tumor microenvironment.

### Example 10. pH-Dependent Anti-CD98 Antibodies

In addition to H15L54, pH-dependent antibodies Ab8332, H15L1 and H15L35, were also identified by the same process. The binding activity, pH dependency, and antibody distribution were investigated in mouse models.

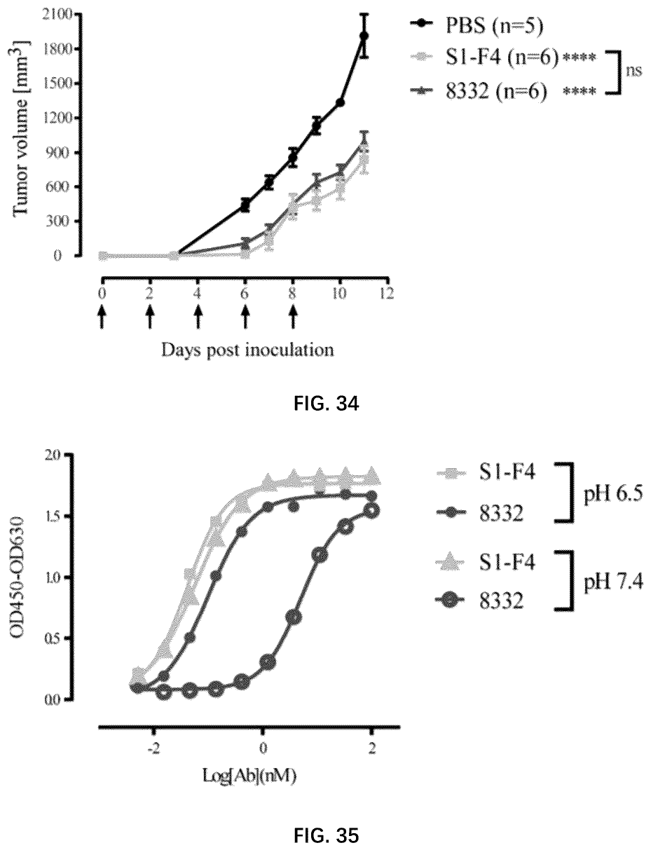

Ab8332 showed a comparable inhibitory effect of tumor growth as S1-F4 in EL4-hCD98 tumor model established in CD98 humanized mice when administered at indicated time (arrows) and at a dosage of 15 mg/kg (FIG. 34).

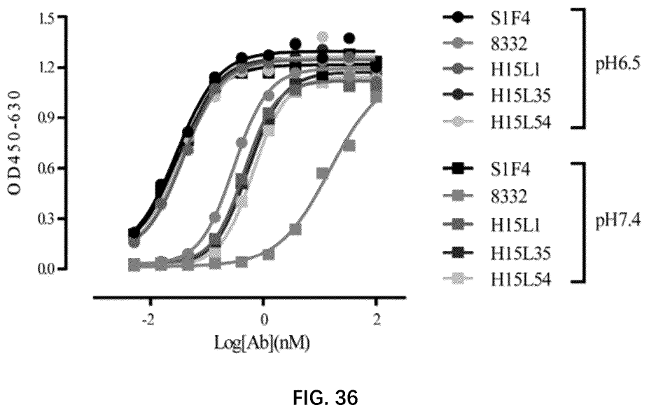

The pH-dependent binding property of Ab8332 was confirmed by using ELISA to detect the binding of S1-F4 and Ab8332 to hCD98 ECD-His₆-Avi-Biotin at pH 6.5 and pH 7.4. As shown in FIG. 35, the EC50 ratio (pH 7.4/6.5) of Ab8332 is as high as 50.7, when S1-F4 did not show any significant difference. H15L1 and H15L35 showed EC50 ratio (pH 7.4/6.5) of 11.8 and 16.0, respectively in ELISA assay (FIG. 36). In addition, the binding of H15L1 and H15L35 to A-431 tumor model detected by FACS also supported a pH-dependent binding (FIG. 28).

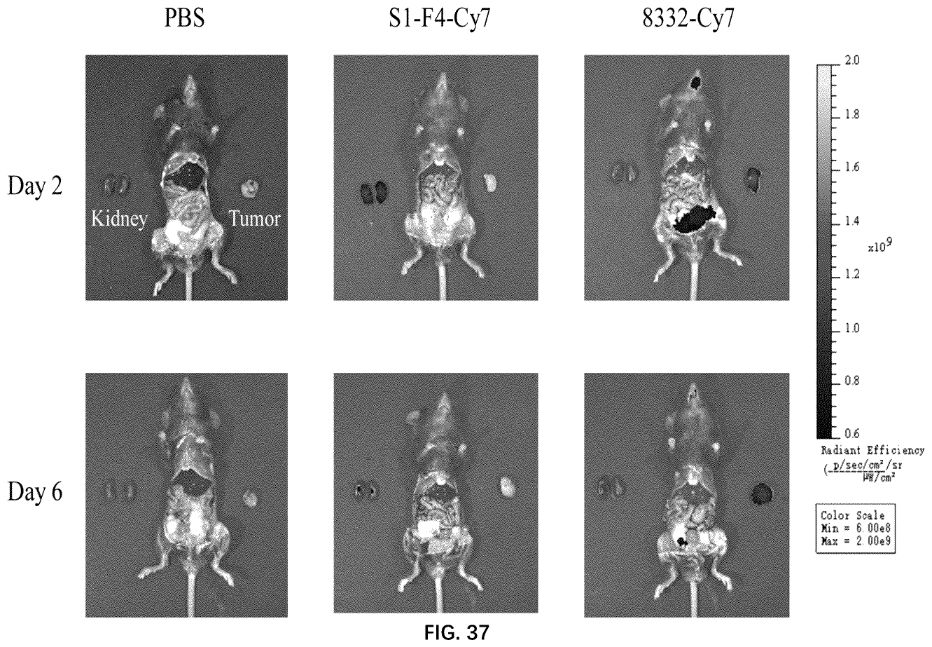

Similar to H15L54, fluorescence imaging of Ab8332-Cy7 in hCD98ECD mice bearing MC38-hCD98 tumor showed its enrichment mainly in tumor rather than in kidneys at day 2 and day 6 after administration (FIG. 37).

### Sequence Listing

Amino acid sequences and nucleotide sequences of the variable regions of S1-F4, H15L54 and Ab8332 can be found in sequence listing with designated SEQ ID NOs based on the following table. CDRs are determined based on Kabat numbering scheme in the present application.

TABLE 3
Sequence Information of S1-F4, H15L1,
H15L35, H15L54 and 8332 anti-CD98 antibodies

| SEQ ID | | SEQ | |
|---|---|---|---|
| No | Description | ID No | Description |
| 10/11/12 | S1-F4 HCDR1/2/3 | 15/16/17 | S1-F4 LCDR1/2/3 |
| 13 | S1-F4 VH amino | 18 | S1-F4 VL amino |
| | acid seq | | acid seq |
| 14 | S1-F4 VH DNA seq | 19 | S1-F4 VL DNA seq |
| 20/21/22 | H15L54 HCDR1/2/3 | 25/26/27 | H15L54 LCDR1/2/3 |
| 23 | H15L54 VH amino | 28 | H15L54 VL amino |
| | acid seq | | acid seq |
| 24 | H15L54 VH DNA seq | 29 | H15L54 VL DNA seq |
| 30/31/32 | 8332 HCDR1/2/3 | 35/36/37 | 8332 LCDR1/2/3 |
| 33 | 8332 VH amino | 38 | 8332 VL amino |
| | acid seq | | acid seq |
| 34 | 8332 VH DNA seq | 39 | 8332 VL DNA seq |
| 40/41/42 | H15L1 HCDR1/2/3 | 45/46/47 | H15L1 LCDR1/2/3 |
| 43 | H15L1 VH amino | 48 | H15L1 VL amino |
| | acid seq | | acid seq |
| 44 | H15L1 VH DNA seq | 49 | H15L1 VL DNA seq |
| 50/51/52 | H15L35 HCDR1/2/3 | 55/56/57 | H15L35 LCDR1/2/3 |
| 53 | H15L35 VH amino | 58 | H15L35 VL amino |
| | acid seq | | acid seq |
| 54 | H15L35 VH DNA seq | 59 | H15L35 VL DNA seq |
| 60/61/62 | HN2-G9 HCDR1/2/3 | 65/66/67 | HN2-G9 LCDR1/2/3 |
| 63 | HN2-G9 VH amino | 68 | HN2-G9 VL amino |
| | acid seq | | acid seq |
| 64 | HN2-G9 VH DNA seq | 69 | HN2-G9 VL DNA seq |
| 70/71/72 | BC8 HCDR1/2/3 | 75/76/77 | BC8 LCDR1/2/3 |
| 73 | BC8 VH amino | 78 | BC8 VL amino |
| | acid seq | | acid seq |
| 74 | BC8 VH DNA seq | 79 | BC8 VL DNA seq |

1. An isolated antibody or an antigen-binding fragment thereof, which binds to human or mouse CD98, wherein the antibody or antigen-binding fragment thereof comprises:
a heavy chain CDR1 (HCDR1) comprising or consisting of an amino acid sequence selected from the group consisting of SEQ ID NOs:10, 20, 30, 40, 50, 60, and 70;
a heavy chain CDR2 (HCDR2) comprising or consisting of an amino acid sequence selected from the group consisting of SEQ ID NOs:11, 21, 31, 41, 51, 61, and 71;
a heavy chain CDR3 (HCDR3) comprising or consisting of an amino acid sequence selected from the group consisting of SEQ ID NOs:12, 22, 32, 42, 52, 62 and 72;
a light chain CDR1 (LCDR1) comprising or consisting of an amino acid sequence selected from the group consisting of SEQ ID NOs:15, 25, 35, 45, 55, 65 and 75;
a light chain CDR2 (LCDR2) comprising or consisting of an amino acid sequence selected from the group consisting of SEQ ID NOs:16, 26, 36, 46, 56, 66 and 76;
a light chain CDR3 (LCDR3) comprising or consisting of an amino acid sequence selected from the group consisting of SEQ ID NOs:17, 27, 37, 47, 57, 67 and 77.

2. The antibody or fragment thereof of claim 1, which comprises any one of following (a) to (g):
(a) HCDR1, HCDR2 and HCDR3 comprising or consisting of an amino acid sequence of SEQ ID NOs:10, 11 and 12, respectively; and LCDR1, LCDR2, LCDR3 comprising or consisting of an amino acid sequence of SEQ ID NOs:15, 16 and 17;
(b) HCDR1, HCDR2 and HCDR3 comprising or consisting of an amino acid sequence of SEQ ID NOs:20, 21 and 22, respectively; and LCDR1, LCDR2, LCDR3 comprising or consisting of an amino acid sequence of SEQ ID NOs:25, 26 and 27;
(c) HCDR1, HCDR2 and HCDR3 comprising or consisting of an amino acid sequence of SEQ ID NOs:30, 31 and 32, respectively; and LCDR1, LCDR2, LCDR3 comprising or consisting of an amino acid sequence of SEQ ID NOs:35, 36 and 37;
(d) HCDR1, HCDR2 and HCDR3 comprising or consisting of an amino acid sequence of SEQ ID NOs:40, 41 and 42, respectively; and LCDR1, LCDR2, LCDR3 comprising or consisting of an amino acid sequence of SEQ ID NOs:45, 46 and 47;
(e) HCDR1, HCDR2 and HCDR3 comprising or consisting of an amino acid sequence of SEQ ID NOs:50, 51 and 52, respectively; and LCDR1, LCDR2, LCDR3 comprising or consisting of an amino acid sequence of SEQ ID NOs:55, 56 and 57;
(f) HCDR1, HCDR2 and HCDR3 comprising or consisting of an amino acid sequence of SEQ ID NOs:60, 61 and 62, respectively; and LCDR1, LCDR2, LCDR3 comprising or consisting of an amino acid sequence of SEQ ID NOs:65, 66 and 67;
(g) HCDR1, HCDR2 and HCDR3 comprising or consisting of an amino acid sequence of SEQ ID NOs:70, 71 and 72, respectively; and LCDR1, LCDR2, LCDR3 comprising or consisting of an amino acid sequence of SEQ ID NOs:75, 76 and 77;
wherein the antibody or fragment thereof comprising any one of following (a) to (f) specifically binds to hCD98, and the antibody or fragment thereof comprising (g) specifically binds to mouse CD98.

3. The antibody or fragment thereof according to claim 1, which comprises:
a heavy chain variable region (VH) comprising an amino acid sequence having at least 80% homology to an amino acid sequence selected from the group consisting of SEQ ID NOs:13, 23, 33, 43, 53, 63 and 73; and/or
a light chain variable region (VL) comprising an amino acid sequence having at least 80% homology to an amino acid sequence selected from the group consisting of SEQ ID NOs:18, 28, 38, 48, 58, 68 and 78.

4. The antibody or fragment thereof according to claim 3, which comprises:
a VH comprising an amino acid sequence selected from the group consisting of SEQ ID NOs:13, 23, 33, 43, 53, 63 and 73, and/or
a VL comprising an amino acid sequence selected from the group consisting of SEQ ID NOs:18, 28, 38, 48, 58, 68 and 78.

5. The antibody or fragment thereof according to claim 3, which comprises any one of following (a) to (g):
(a) a VH comprising or consisting of an amino acid sequence of SEQ ID NO:13, and a VL comprising or consisting of an amino acid sequence of SEQ ID NO:18;
(b) a VH comprising or consisting of an amino acid sequence of SEQ ID NO:23, and a VL comprising or consisting of an amino acid sequence of SEQ ID NO:28;
(c) a VH comprising or consisting of an amino acid sequence of SEQ ID NO:33, and a VL comprising or consisting of an amino acid sequence of SEQ ID NO:38;
(d) a VH comprising or consisting of an amino acid sequence of SEQ ID NO:43, and a VL comprising or consisting of an amino acid sequence of SEQ ID NO:48;
(e) a VH comprising or consisting of an amino acid sequence of SEQ ID NO:53, and a VL comprising or consisting of an amino acid sequence of SEQ ID NO:58;
(f) a VH comprising or consisting of an amino acid sequence of SEQ ID NO:63, and a VL comprising or consisting of an amino acid sequence of SEQ ID NO:68;
(g) a VH comprising or consisting of an amino acid sequence of SEQ ID NO:73, and a VL comprising or consisting of an amino acid sequence of SEQ ID NO:78;
wherein the antibody or fragment thereof comprising any one of following (a) to (f) specifically binds to hCD98, and the antibody or fragment thereof comprising (g) specifically binds to mouse CD98.

6. The antibody or fragment thereof according to claim 1, which has pH dependence in binding to hCD98, wherein the binding activity of the antibody or fragment thereof at acidic pH is higher than the binding activity at neutral pH.

7. The antibody or fragment thereof according to claim 6, wherein the EC50 of the antibody or fragment thereof at acidic pH is at least 2-fold, 3-fold, 5-fold, 10-fold, 15-fold, 20-fold, 30-fold, 40-fold, 50-fold lower than the EC50 at neutral pH when measuring the binding of the antibody to hCD98 when the binding activity is measured by ELISA assay.

8. The antibody or fragment thereof according to claim 6, wherein the acidic pH is 5.8, 5.9, 6.0, 6.1, 6.2, 6.3, 6.4, 6.5, 6.6, 6.7 or 6.8, and the neutral pH is 7.0, 7.1, 7.2, 7.3, 7.4, 7.5 or 7.6, preferably the acidic pH is 6.5 and the neutral pH is 7.4.

9. An isolated polynucleotide encoding the antibody or fragment thereof of claim 1.

10. An expression vector comprising the isolated polynucleotide according to claim 9.

11. A host cell comprising the expression vector according to claim 10.

12. A composition comprising the antibody or fragment thereof of claim 1 and a pharmaceutically acceptable carrier.

13. A method of reducing tumors, inhibiting the growth of tumor cells, treating a cancer or preventing recurrence of a cancer in a subject in need thereof, comprising administering to the subject a therapeutically effective amount of the antibody or fragment thereof according to claim 1.

14. The method of claim 13, wherein the tumor or cancer is CD98-expressing tumor or cancer.

15. The method of claim 13, wherein the tumor or cancer has a microenvironment having acidic pH, e.g. pH 7.0, 6.9, 6.8, 6.7, 6.6, 6.5, 6.4, 6.3, 6.2, 6.1, 6.0 or lower, and the normal physiological pH of the subject is around pH 7.4.

16. The method of claim 13, wherein the tumor or cancer is selected from the group consisting of lymphoma, acute promyelocytic leukemia, hepatocellular carcinoma, pancreatic adenocarcinoma, pancreatic epithelioid carcinoma, breast adenocarcinoma, colorectal adenocarcinoma, skin epidermoid carcinoma, melanoma, fibrosarcoma, non-small cell lung cancer, gastric cancer, acute myeloid leukemia, glioma, tongue cancer, hypopharyngeal squamous cell carcinoma, cholangiocarcinoma, osteomalacia, osteosarcoma, renal cancer, and neuroblastoma.

17. A method of reducing tumors, inhibiting the growth of tumor cells, treating a cancer or preventing recurrence of a cancer in a subject in need thereof, comprising administering to the subject a therapeutically effective amount of the composition of claim 12.

18. The method of claim 17, wherein the tumor or cancer is CD98-expressing tumor or cancer.

19. The method of claim 17, wherein the tumor or cancer has a microenvironment having acidic pH, e.g. pH 7.0, 6.9, 6.8, 6.7, 6.6, 6.5, 6.4, 6.3, 6.2, 6.1, 6.0 or lower, and the normal physiological pH of the subject is around pH 7.4.

20. The method of claim 17, wherein the tumor or cancer is selected from the group consisting of lymphoma, acute promyelocytic leukemia, hepatocellular carcinoma, pancreatic adenocarcinoma, pancreatic epithelioid carcinoma, breast adenocarcinoma, colorectal adenocarcinoma, skin epidermoid carcinoma, melanoma, fibrosarcoma, non-small cell lung cancer, gastric cancer, acute myeloid leukemia, glioma, tongue cancer, hypopharyngeal squamous cell carcinoma, cholangiocarcinoma, osteomalacia, osteosarcoma, renal cancer, and neuroblastoma.


SEQUENCE LISTING (79 sequences)

SEQ ID NO: 1 - DNA, 23 bp, Artificial Sequence
  Small guided RNA targeting CD98
ccgcgctgccgtgagctgcctgt

SEQ ID NO: 2 - DNA, 20 bp, Artificial Sequence
  Y1-F primer
gggtcaagttcaccggctta

SEQ ID NO: 3 - DNA, 20 bp, Artificial Sequence
  Y1-R primer
ctcaggtaatcgagacgccc

SEQ ID NO: 4 - DNA, 20 bp, Artificial Sequence
  WT1-F primer
gtgaagatcaaggtggcgga

SEQ ID NO: 5 - DNA, 20 bp, Artificial Sequence
  WT1-R primer
aaaaagcagcagaatgcccg

SEQ ID NO: 6 - DNA, 20 bp, Artificial Sequence
  MS2-F primer
gggtcaagttcaccggctta

SEQ ID NO: 7 - DNA, 19 bp, Artificial Sequence
  MS2-R primer
ctgggccagaatgccaaac

SEQ ID NO: 8 - DNA, 20 bp, Artificial Sequence
  IF-1 primer
gtgtaagccccgaggagaat

SEQ ID NO: 9 - protein, 529 aa, Homo sapiens
MSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 10 - protein, 5 aa, Artificial Sequence
  S1-F4 HCDR1
SYYMH

SEQ ID NO: 11 - protein, 17 aa, Artificial Sequence
  S1-F4 HCDR2
IINPSGGSTSYAQKFQG

SEQ ID NO: 12 - protein, 16 aa, Artificial Sequence
  S1-F4 HCDR3
GYYDILTGSRPIFFDI

SEQ ID NO: 13 - protein, 125 aa, Artificial Sequence
  S1-F4 VH amino acid seq
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGYYDILTGSRPIFFDIWGQGTMVTVSS

SEQ ID NO: 14 - DNA, 375 bp, Artificial Sequence
  S1-F4 VH DNA seq
caggtccagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagggggtattacgatattttgactggttcccgacctattttttttgatatctggggccaagggacaatggtcaccgtctcttca

SEQ ID NO: 15 - protein, 17 aa, Artificial Sequence
  S1-F4 LCDR1
KSSQSVLYSSNNKNYLA

SEQ ID NO: 16 - protein, 7 aa, Artificial Sequence
  S1-F4 LCDR2
WASTRES

SEQ ID NO: 17 - protein, 9 aa, Artificial Sequence
  S1-F4 LCDR3
QQYYNTPIT

SEQ ID NO: 18 - protein, 113 aa, Artificial Sequence
  S1-F4 VL amino acid seq
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYNTPITFGQGTRLEIK

SEQ ID NO: 19 - DNA, 339 bp, Artificial Sequence
  S1-F4 VL DNA seq
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagttgctcatttactgggcatctacccgggaatccggggtccctgaccgattcagtggcagcgggtctggaacagatttcactctcaccatcagcagcctgcaggctgaagatgtggcagtttattactgtcagcaatattataatactccgatcaccttcggccaagggacacgactggagattaaa

SEQ ID NO: 20 - protein, 5 aa, Artificial Sequence
  H15L54 HCDR1
SYQMH

SEQ ID NO: 21 - protein, 17 aa, Artificial Sequence
  H15L54 HCDR2
IINDKHGSTSYAQKFQG

SEQ ID NO: 22 - protein, 16 aa, Artificial Sequence
  H15L54 HCDR3
GYEDSNTHQHPIFFDI

SEQ ID NO: 23 - protein, 125 aa, Artificial Sequence
  H15L54 VH amino acid seq
QVQLVQSGAEVKKPGASVKVSCKASGYTFTSYQMHWVRQAPGQGLEWMGIINDKHGSTSYAQKFQGRVTMTRDTSTSTVYMELSSLRSEDTAVYYCARGYEDSNTHQHPIFFDIWGQGTMVTVSS

SEQ ID NO: 24 - DNA, 375 bp, Artificial Sequence
  H15L54 VH DNA seq
caggtccagctggtgcagtctggggctgaggtgaagaagcctggggcctcagtgaaggtttcctgcaaggcatctggatacaccttcaccagctaccagatgcactgggtgcgacaggcccctggacaagggcttgagtggatgggaataatcaacgacaagcacggtagcacaagctacgcacagaagttccagggcagagtcaccatgaccagggacacgtccacgagcacagtctacatggagctgagcagcctgagatctgaggacacggccgtgtattactgtgcgagggggtatgaggatagcaacactcaccagcacccgattttttttgatatctggggccaagggacaatggtcaccgtctcttca

SEQ ID NO: 25 - protein, 17 aa, Artificial Sequence
  H15L54 LCDR1
KSSQSVLYRHNNKNYLA

SEQ ID NO: 26 - protein, 7 aa, Artificial Sequence
  H15L54 LCDR2
WASTSES

SEQ ID NO: 27 - protein, 9 aa, Artificial Sequence
  H15L54 LCDR3
QQYYNTPIT

SEQ ID NO: 28 - protein, 113 aa, Artificial Sequence
  H15L54 VL amino acid seq
DIVMTQSPDSLAVSLGERATINCKSSQSVLYRHNNKNYLAWYQQKPGQPPKLLIYWASTSESGVPDRFSGSGSGTDFTLTISSLQAEDVAVYYCQQYYNTPITFGQGTRLEIK

SEQ ID NO: 29 - DNA, 339 bp, Artificial Sequence
  H15L54 VL DNA seq
gacatcgtgatgacccagtctccagactccctggctgtgtctctgggcgagagggccaccatcaactgcaagtccagccagagtgtgctgtacaggcacaacaataagaactacttagcttggtaccagcagaaaccaggacagcctcctaagttgctcatttactgggcatctaccagcgaatccggggtccctgaccgattcagtggcagcgggtctggaacagatttcactctcaccatcagcagcctgcaggctgaagatgtggcagtttattactgtcagcaatattataatactccgatcaccttcggccaagggacacgactggagattaaa

SEQ ID NO: 30 - protein, 5 aa, Artificial Sequence
  8332 HCDR1
SYQMH

SEQ ID NO: 31 - protein, 17 aa, Artificial Sequence
  8332 HCDR2
IINPSGGSTSYAQKFQG

SEQ ID NO: 32 - protein, 16 aa, Artificial Sequence
  8332 HCDR3
GYEDSNTHQHPIFFDI

SEQ ID NO: 33 - protein, 125 aa, Artificial Sequence
  8332 VH amino acid seq
QVQLVQSGAEVKKPGASVKVSCKASGYTFTSYQMHWVRQAPGQGLEWMGIINPSGGSTSYAQKFQGRVTMTRDTSTSTVYMELSSLRSEDTAVYYCARGYEDSNTHQHPIFFDIWGQGTMVTVSS

SEQ ID NO: 34 - DNA, 375 bp, Artificial Sequence
  8332 VH DNA seq
caggtccagctggtgcagtctggggctgaggtgaagaagcctggggcctcagtgaaggtttcctgcaaggcatctggatacaccttcaccagctaccagatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagggggtatgaggatagcaacactcaccagcacccgattttttttgatatctggggccaagggacaatggtcaccgtctcttca

SEQ ID NO: 35 - protein, 17 aa, Artificial Sequence
  8332 LCDR1
KSSQSVLYSSNNKNYLA

SEQ ID NO: 36 - protein, 7 aa, Artificial Sequence
  8332 LCDR2
WASTRES

SEQ ID NO: 37 - protein, 9 aa, Artificial Sequence
  8332 LCDR3
QQYYNTPIT

SEQ ID NO: 38 - protein, 113 aa, Artificial Sequence; identical to SEQ ID NO: 18
  8332 VL amino acid seq

SEQ ID NO: 39 - DNA, 339 bp, Artificial Sequence; identical to SEQ ID NO: 19
  8332 VL DNA seq

SEQ ID NO: 40 - protein, 5 aa, Artificial Sequence
  H15L1 HCDR1
SYQMH

SEQ ID NO: 41 - protein, 17 aa, Artificial Sequence
  H15L1 HCDR2
IINDKHGSTSYAQKFQG

SEQ ID NO: 42 - protein, 16 aa, Artificial Sequence
  H15L1 HCDR3
GYEDSNTHQHPIFFDI

SEQ ID NO: 43 - protein, 125 aa, Artificial Sequence; identical to SEQ ID NO: 23
  H15L1 VH amino acid seq

SEQ ID NO: 44 - DNA, 375 bp, Artificial Sequence; identical to SEQ ID NO: 24
  H15L1 VH DNA seq

SEQ ID NO: 45 - protein, 17 aa, Artificial Sequence
  H15L1 LCDR1
KSSQSVLYRHNNKNYLA

SEQ ID NO: 46 - protein, 7 aa, Artificial Sequence
  H15L1 LCDR2
WASTRES

SEQ ID NO: 47 - protein, 9 aa, Artificial Sequence
  H15L1 LCDR3
QQYYNTPIT

SEQ ID NO: 48 - protein, 113 aa, Artificial Sequence
  H15L1 VL amino acid seq
DIVMTQSPDSLAVSLGERATINCKSSQSVLYRHNNKNYLAWYQQKPGQPPKLLIYWASTRESGVPDRFSGSGSGTDFTLTISSLQAEDVAVYYCQQYYNTPITFGQGTRLEIK

SEQ ID NO: 49 - DNA, 339 bp, Artificial Sequence
  H15L1 VL DNA seq
gacatcgtgatgacccagtctccagactccctggctgtgtctctgggcgagagggccaccatcaactgcaagtccagccagagtgtgctgtacaggcacaacaataagaactacttagcttggtaccagcagaaaccaggacagcctcctaagttgctcatttactgggcatctacccgggaatccggggtccctgaccgattcagtggcagcgggtctggaacagatttcactctcaccatcagcagcctgcaggctgaagatgtggcagtttattactgtcagcaatattataatactccgatcaccttcggccaagggacacgactggagattaaa

SEQ ID NO: 50 - protein, 5 aa, Artificial Sequence
  H15L35 HCDR1
SYQMH

SEQ ID NO: 51 - protein, 17 aa, Artificial Sequence
  H15L35 HCDR2
IINDKHGSTSYAQKFQG

SEQ ID NO: 52 - protein, 16 aa, Artificial Sequence
  H15L35 HCDR3
GYEDSNTHQHPIFFDI

SEQ ID NO: 53 - protein, 125 aa, Artificial Sequence; identical to SEQ ID NO: 23
  H15L35 VH amino acid seq

SEQ ID NO: 54 - DNA, 375 bp, Artificial Sequence; identical to SEQ ID NO: 24
  H15L35 VH DNA seq

SEQ ID NO: 55 - protein, 17 aa, Artificial Sequence
  H15L35 LCDR1
KSSQSVLYRHNNNNYLA

SEQ ID NO: 56 - protein, 7 aa, Artificial Sequence
  H15L35 LCDR2
WASTSES

SEQ ID NO: 57 - protein, 9 aa, Artificial Sequence
  H15L35 LCDR3
QQYYNTPIT

SEQ ID NO: 58 - protein, 113 aa, Artificial Sequence
  H15L35 VL amino acid seq
DIVMTQSPDSLAVSLGERATINCKSSQSVLYRHNNNNYLAWYQQKPGQPPKLLIYWASTSESGVPDRFSGSGSGTDFTLTISSLQAEDVAVYYCQQYYNTPITFGQGTRLEIK

SEQ ID NO: 59 - DNA, 339 bp, Artificial Sequence
  H15L35 VL DNA seq
gacatcgtgatgacccagtctccagactccctggctgtgtctctgggcgagagggccaccatcaactgcaagtccagccagagtgtgctgtacaggcacaacaataacaactacttagcttggtaccagcagaaaccaggacagcctcctaagttgctcatttactgggcatctaccagcgaatccggggtccctgaccgattcagtggcagcgggtctggaacagatttcactctcaccatcagcagcctgcaggctgaagatgtggcagtttattactgtcagcaatattataatactccgatcaccttcggccaagggacacgactggagattaaa

SEQ ID NO: 60 - protein, 5 aa, Artificial Sequence
  HN2-G9 HCDR1
SYYMH

SEQ ID NO: 61 - protein, 17 aa, Artificial Sequence
  HN2-G9 HCDR2
IINPSGGSTSYAQKFQG

SEQ ID NO: 62 - protein, 14 aa, Artificial Sequence
  HN2-G9 HCDR3
GYCSGGRCYSDFQH

SEQ ID NO: 63 - protein, 123 aa, Artificial Sequence
  HN2-G9 VH amino acid seq
QVQLVQSGAEVKKPGSSVKVSCKASGYTFTSYYMHWVRQAPGQGLEWMGIINPSGGSTSYAQKFQGRVTMTRDTSTSTVYMELSSLRSEDTAVYFCARGYCSGGRCYSDFQHWGQGTLVTVSS

SEQ ID NO: 64 - DNA, 369 bp, Artificial Sequence
  HN2-G9 VH DNA seq
caggtgcagctggtgcagtctggggctgaggtgaagaagcctgggtcctcggtgaaggtctcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtatttctgtgcgagaggatattgtagtggcggtaggtgctactctgacttccagcactggggccagggcaccctggtcaccgtctcctca

SEQ ID NO: 65 - protein, 17 aa, Artificial Sequence
  HN2-G9 LCDR1
KSSQSVLYSSNNKNYLA

SEQ ID NO: 66 - protein, 7 aa, Artificial Sequence
  HN2-G9 LCDR2
WASTRES

SEQ ID NO: 67 - protein, 9 aa, Artificial Sequence
  HN2-G9 LCDR3
QQYYNTPIT

SEQ ID NO: 68 - protein, 113 aa, Artificial Sequence; identical to SEQ ID NO: 18
  HN2-G9 VL amino acid seq

SEQ ID NO: 69 - DNA, 339 bp, Artificial Sequence; identical to SEQ ID NO: 19
  HN2-G9 VL DNA seq

SEQ ID NO: 70 - protein, 5 aa, Artificial Sequence
  BC8 HCDR1
NNYMS

SEQ ID NO: 71 - protein, 16 aa, Artificial Sequence
  BC8 HCDR2
VIYSGGKTYYADSAKG

SEQ ID NO: 72 - protein, 9 aa, Artificial Sequence
  BC8 HCDR3
VSGLGWFDP

SEQ ID NO: 73 - protein, 117 aa, Artificial Sequence
  BC8 VH amino acid seq
EVQLVESGGGVVQPGRSLRLSCAASGFTVSNNYMSWVRQAPGKGLEWVSVIYSGGKTYYADSAKGRFTISRDNSKNTVFLQMNSLRVEDTAVYYCARVSGLGWFDPWGQGTLVTVSS

SEQ ID NO: 74 - DNA, 351 bp, Artificial Sequence
  BC8 VH DNA seq
gaggtgcagctggtggagtctgggggaggcgtggtccagcctgggaggtccctgagactctcctgtgcagcctctggattcaccgtcagtaataattacatgagctgggtccgccaggctccagggaagggactggagtgggtctcagttatttatagtggtgggaaaacatactacgcggactccgcgaagggcagattcaccatctccagagacaattccaagaacacggtgtttcttcaaatgaacagcctgagagtcgaggacacggctgtgtattactgtgcgagagtttcaggccttggctggttcgacccctggggccagggaaccctggtcaccgtctcctca

SEQ ID NO: 75 - protein, 13 aa, Artificial Sequence
  BC8 LCDR
SGSSSNIGSNYVY

SEQ ID NO: 76 - protein, 7 aa, Artificial Sequence
  BC8 LCDR2
RNNQRPS

SEQ ID NO: 77 - protein, 11 aa, Artificial Sequence
  BC8 LCDR3
AAWDDSLSGWV

SEQ ID NO: 78 - protein, 110 aa, Artificial Sequence
  BC8 VL amino acid seq
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGWVFGGGTKLTVL

SEQ ID NO: 79 - DNA, 330 bp, Artificial Sequence
  BC8 VL DNA seq
cagtctgtgttgacgcagccgccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggttgggtgttcggcggagggaccaagctgaccgtccta